(19) United States
(12) Patent Application Publication
(10) Pub. No.: US 2026/0311855 A1
(43) Pub. Date: Oct. 8, 2026

(54) AGENTIC ARTIFICIAL INTELLIGENCE CLINICAL INTAKE SYSTEM WITH CULTURALLY ADAPTIVE VIRTUAL AGENTS, DEFENSE-IN-DEPTH SAFETY ENFORCEMENT, DOMAIN TRACKING STATE MACHINE, AND AUTOMATED CLINICAL DOCUMENTATION

(71) Applicants: James B. Herrera, Carmichael, CA (US); Angela M. Marshall, Silver Spring, MD (US); Barbara J. Brown, Annapolis, MD (US); Charles Senteio, North Brunswick, NY (US); Isaiah Marshall, Silver Spring, MD (US)

(72) Inventors: James B. Herrera, Carmichael, CA (US); Angela M. Marshall, Silver Spring, MD (US); Barbara J. Brown, Annapolis, MD (US); Charles Senteio, North Brunswick, NY (US); Isaiah Marshall, Silver Spring, MD (US)

(21) Appl. No.: 19/637,845

(22) Filed: Apr. 2, 2026

Related U.S. Application Data
(60) Provisional application No. 63/782,756, filed on Apr. 3, 2025.

(51) Int. Cl. G16H 40/20; G06F 16/3329; G06F 40/157; G16H 10/60

(52) U.S. Cl. CPC G16H 40/20; G06F 16/33295; G06F 40/157; G16H 10/60

(57) ABSTRACT

An agentic artificial intelligence intake system conducts conversational user interviews using culturally adaptive virtual agents. The system dynamically selects assessment frameworks from a plurality of stored frameworks based on keyword analysis of an initial user input and generates contextual questions through unified API calls with guardrails derived from a domain tracking state machine. Defense-in-depth safety enforcement provides dual-layer detection using AI semantic classification and deterministic pattern matching across a plurality of safety categories, with hard completion gates preventing session termination until mandatory safety domains are addressed. The domain tracking state machine tracks domain coverage through discrete states comprising NOT_STARTED, ASKED, ANSWERED, INFERRED, SKIPPED, and BLOCKED, with enforced state transitions. Virtual agent personalities are layered on mandatory base guardrails enforcing safety protocols and framework compliance. Context-aware empathy injection with prefix stripping enables redundancy detection while maintaining conversational flow. The system generates structured output documents through automated language transformation converting conversational user speech into domain-appropriate formal terminology. An adaptive readability subsystem personalizes communication complexity to match each user's demonstrated comprehension level while preserving all safety enforcement requirements.

### CROSS-REFERENCE TO RELATED APPLICATIONS

This application claims the benefit of U.S. Provisional Application No. 63/782,756, filed Apr. 3, 2025, entitled “Culturally Adaptive Medical History Collection System with Personalized Virtual Agent,” the entire disclosure of which is hereby incorporated by reference in its entirety.

### FIELD

The present disclosure relates to artificial intelligence systems for healthcare, and more particularly to conversational AI systems that conduct clinical patient intake interviews using agentic reasoning, culturally adaptive virtual agent personalities, adaptive language communication, mandatory safety enforcement with defense-in-depth detection, clinical domain coverage tracking through explicit state machines, automated clinical documentation generation with clinical language transformation, and automatic recommendation of appropriate clinical screenings based on aggregated symptom data.

### BACKGROUND

Patient intake, the process of gathering medical history, symptoms, and relevant clinical information before a healthcare encounter, may be fundamental to quality care. Traditional intake may rely on paper forms or rigid electronic questionnaires that may fail to capture the nuanced, conversational nature of clinical interviews. These traditional methods may suffer from several well-documented limitations: time constraints on healthcare providers leading to abbreviated history-taking; cultural and communication barriers causing incomplete information capture; lack of adaptive questioning resulting in either over-questioning or dangerous omissions; and no systematic mechanism for ensuring safety-critical screenings are completed.

Recent advances in large language models (LLMs) have enabled more natural conversational interfaces for healthcare. However, applying general-purpose LLMs to clinical intake may introduce new technical challenges that existing systems have not adequately addressed. Several prior art systems have attempted to address aspects of clinical intake automation, but majority are doctor-facing, as it may assist the physician during an encounter already in progress, rather than patient-facing. It may not conduct patient intake interviews, may not provide virtual agent personality selection, may not implement domain tracking state machines, and may not generate clinical documentation from conversational intake data. Other prior art systems in the electronic health record (EHR) and patient engagement space may provide digital intake questionnaires. However, these systems may present rigid, predetermined question sets without adaptive intelligence. They may not dynamically select clinical assessment frameworks based on chief complaints, may not implement safety enforcement gates, and may not provide conversational AI interfaces. Academic literature describes various applications of natural language processing (NLP) to clinical documentation, including automated SOAP note generation. However, these systems may operate on existing clinical encounter transcripts rather than conducting the intake interview itself, and may not address the unique safety challenges of AI-patient interaction.

Generally, existing electronic intake systems may present identical questions to all patients regardless of their specific complaints. A patient presenting for a routine physical may receive the same lengthy questionnaire as one presenting for acute chest pain. Prior systems that attempt adaptation may use simple branching logic that may not handle the complexity of real clinical reasoning where multiple symptoms interact and context matters.

Furthermore, existing intake systems may fail to account for the diverse cultural, educational, and linguistic backgrounds of patients. A system that uses uniform medical terminology may confuse patients with lower health literacy, while one that oversimplifies may frustrate medically knowledgeable patients. No prior system may provide culturally specific virtual agent personalities that patients can select to feel more comfortable during the intake interview, nor dynamically adjust communication style based on observed patient vocabulary and communication patterns.

There is a need for an improved client intake and screening system. The present disclosure addresses these and other challenges in an integrated architecture.

### BRIEF SUMMARY

The present disclosure provides an agentic intake system comprising multiple interconnected subsystems. In one aspect, the present disclosure provides a computer-implemented method for conducting an agentic intake session comprising: dynamically selecting assessment frameworks based on initial user input keyword analysis; generating guardrails from a domain tracking state machine; enforcing mandatory safety screenings through hard completion gates; applying defense-in-depth dual-layer safety detection with corroboration requirements; generating structured output documents with language transformation; and post-processing AI output through domain-appropriate safety filters.

In another aspect, the present disclosure provides an agentic intake engine that may dynamically select frameworks, may generate contextual questions through unified API calls, and may track progress through explicit domain state machines with six discrete states. In another aspect, the present disclosure provides defense-in-depth safety enforcement comprising: a primary AI semantic classifier with enumerated safety levels and categories; a secondary deterministic pattern detector scanning twelve safety categories including suicidal ideation, self-harm, homicidal ideation, psychosis, severe depression, substance crisis, abuse disclosure, domestic violence, eating disorder crisis, mania crisis, elder abuse, and trafficking/exploitation; hard completion gates; and corroboration requirements for AI-reported safety coverage. A safety category for medical emergencies may include cardiac symptoms, stroke symptoms, severe allergic reaction, syncope, severe abdominal pain, and heavy vaginal bleeding.

In another aspect, the present disclosure provides an agentic intake engine that may dynamically select frameworks from a framework repository, may generate contextual questions through unified API calls, and may track progress through explicit domain state machines with six discrete states. In another aspect, the present disclosure provides defense-in-depth safety enforcement comprising: a primary AI semantic classifier with enumerated safety levels and categories; a secondary deterministic pattern detector scanning a plurality of safety categories; hard completion gates; and corroboration requirements for AI-reported safety coverage. In some embodiments, safety categories may include crisis conditions applicable to the deployment domain, such as safety-critical disclosures in healthcare, legal, financial compliance, or human resources intake sessions.

In another further aspect, the present disclosure provides a domain tracking state machine with enforced transitions, two-pass coverage evaluation, and prompt injection of domain status. In another aspect, the present disclosure provides proxy and caregiver detection classifying scenarios into pediatric, geriatric, spouse, caregiver, and guardian categories with automatic question phrasing adjustment. In another aspect, the present disclosure provides automated clinical document generation producing SOAP notes and 21-section behavioral health reports with clinical language transformation.

In another further aspect, the present disclosure provides a domain tracking state machine with enforced transitions, two-pass coverage evaluation, and prompt injection of domain status. In another aspect, the present disclosure provides proxy and third-party detection classifying scenarios into a plurality of relationship categories with automatic question phrasing adjustment. In another aspect, the present disclosure provides automated structured document generation producing domain-appropriate output documents with language transformation converting conversational user speech into formal terminology applicable to the deployment domain, such as medical terminology for healthcare intake sessions, human resources terminology for employment onboarding sessions, legal terminology for legal intake sessions, or compliance terminology for regulatory intake sessions.

In yet another aspect, the present disclosure provides context-aware empathy injection with empathy prefix stripping for redundancy detection. In another aspect, the present disclosure provides real-time structured data reconciliation with extraction, reconciliation, completeness evaluation, and follow-up probes applicable across deployment domains. In another aspect, the present disclosure provides a multi-phase intake architecture with a plurality of defined phases and context bridging between phases.

In another aspect, the present disclosure provides virtual agent personalities layered on mandatory base guardrails. In another aspect, the present disclosure provides domain-specific code and classification suggestions from conversation analysis. In another aspect, the present disclosure provides screening recommendations from checklist analysis with severity-based thresholds. In another aspect, the present disclosure provides an output safety filter using pattern matching. In another aspect, the present disclosure provides stalled coverage detection redirecting AI when conversations loop without covering new required domains.

In yet another aspect, the present disclosure provides external records context injection through standard interoperability interfaces, such as FHIR R4 and HL7 v2 interfaces for healthcare deployments. In another aspect, the present disclosure provides dual-source flag detection with mandatory follow-up questions. In another aspect, the present disclosure provides an adaptive readability and linguistic personalization subsystem that may dynamically match the linguistic complexity of AI-generated output to an individual user's demonstrated reading level and communication preferences.

### BRIEF DESCRIPTION OF THE DRAWINGS

The accompanying drawings, which are incorporated in and constitute a part of this specification, illustrate embodiments of the present disclosure and, together with the description, explain certain principles of the present disclosure. The drawings are provided for purposes of illustration only and merely depict example embodiments. These drawings are provided to facilitate the reader's understanding of the present disclosure and shall not be considered limiting of the breadth, scope, or applicability of the present disclosure. It will be appreciated that for simplicity and clarity of illustration, elements shown in the figures have not necessarily been drawn to scale. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose. Furthermore, the drawings may contain text or captions that may explain certain embodiments of the present disclosure. This text is included for illustrative, non-limiting, explanatory purposes of certain embodiments detailed in the present disclosure. The present disclosure, in accordance with one or more various embodiments, is described in detail with reference to the following figures.

FIG. 1A is a high-level system architecture diagram of the integrated clinical intake system (100A).

FIG. 1B is a detailed view of the agentic clinical intake engine components.

FIG. 1C is a flowchart of the unified AI response generation process.

FIG. 1D is a high-level system architecture diagram of the integrated clinical intake system (100D).

FIG. 2 is a sequence diagram of a complete conversation turn with system components in accordance with the invention.

FIG. 3 is an architecture diagram of the platform in accordance with the invention.

FIG. 4 is a flowchart of a method of operation in accordance with the invention.

FIG. 5 is a decision tree in accordance with the invention.

FIG. 6 is a computing device in accordance with the invention.

### DETAILED DESCRIPTION

The following detailed description is presented to enable any person skilled in the art to make and use the present disclosure. For purposes of explanation, specific details are set forth to provide a thorough understanding of the present disclosure. However, these specific details are not required to practice the present disclosure, and in other instances, well-known structures and devices are shown in block diagram form in order to avoid obscuring the present disclosure. The following detailed description is, therefore, not to be taken in a limiting sense. The present disclosure is defined by the appended claims.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

The present disclosure will now be described more fully hereinafter with reference to the accompanying drawings, in which example embodiments of the present disclosure are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. It is to be understood that the present disclosure is not limited to the embodiments described herein and illustrated in the drawings; rather, the skilled person will recognize that many changes and modifications may be made within the scope of the appended claims.

The following description is merely illustrative in nature and is in no way intended to limit the disclosure, its application, or uses. For purposes of clarity, the same reference numbers may be used in the drawings to identify similar elements. As used herein, the phrase “at least one of” A, B, and C should be construed to mean a logical (A or B or C), using a non-exclusive logical OR. It should be understood that steps within a method may be executed in different order without altering the principles of the present disclosure. Throughout this specification and the claims, the terms “comprise,” “comprising,” “include,” “including,” and the like are to be understood to imply the inclusion of stated elements but not the exclusion of any other elements. No claim element herein is to be construed under the provisions of 35 U.S.C. § 112(f) unless the element is expressly recited using the exact phrase “means for.” Where a method claim does not specifically state steps are to be limited to a specific order, no order is inferred. While aspects may be described and claimed in a particular statutory class, each aspect may be described and claimed in any statutory class.

The embodiments described herein are provided by way of example only. Numerous variations, changes, and substitutions may occur to those skilled in the art without departing from the present disclosure. It should be understood that various alternatives to the embodiments described herein may be employed. The features of any one embodiment described herein may be combined with features of any other embodiment. Features may be added, removed, or substituted without departing from the scope of the present disclosure. The present disclosure is applicable in many different fields and contexts beyond those specifically described herein. Nothing in this disclosure should be construed as limiting the present disclosure to any particular field, application, industry, or use case. The examples provided herein are illustrative of only some of the possible embodiments of many, and the present disclosure contemplates numerous variations, modifications, and adaptations that will become apparent to those skilled in the art upon reading and understanding the foregoing description.

Before the present articles, systems, apparatuses, and methods are disclosed and described, it is to be understood that they are not limited to specific methods unless otherwise specified, or to particular materials unless otherwise specified, as such can vary. It is also to be understood that the terminology used herein is for the purpose of describing particular aspects only and is not intended to be limiting. Although any methods and materials similar or equivalent to those described herein may be used in the practice or testing of the present disclosure, example methods and materials are now described. Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect.

### Overview of Integrated System Architecture

The following detailed description presents various embodiments of systems and methods for conducting agentic intake sessions using conversational artificial intelligence. The embodiments described herein provide technical solutions for deploying AI-driven conversational systems across a plurality of deployment domains, including but not limited to healthcare, human resources, legal services, financial compliance, and regulatory intake, with culturally adaptive virtual agents, defense-in-depth safety enforcement, and automated structured document generation.

In some embodiments, the system implements hard completion gates that prevent interview termination until mandatory safety domains have been addressed. These completion gates operate independently of AI decisions, patient requests, or configuration changes, ensuring that critical screenings such as suicide risk assessment occur before the system allows the interview to conclude.

Certain embodiments employ redundant detection mechanisms that implement independent verification layers to analyze patient responses for safety-critical information. Rather than relying on a single language model for both question generation and safety detection, these embodiments use separate detection pathways to identify concerning disclosures, including passive expressions of suicidal ideation. The system verifies domain coverage through actual question-and-answer exchanges rather than accepting AI self-reports of completion. Some embodiments include content filtering mechanisms that intercept and modify AI-generated responses before presenting them to patients. These filters prevent the system from generating medical advice, diagnoses, or treatment recommendations, providing a technical safeguard beyond prompt engineering alone.

Various embodiments provide automated documentation generation capabilities that transform conversational exchanges into structured clinical notes. The system produces documentation that balances clinical precision with contextual nuance captured during natural conversation, eliminating the need for manual reformatting of verbatim transcripts or rigid template-based outputs.

Certain embodiments implement semantic redundancy detection that analyzes the semantic content of questions to identify and prevent repetitive questioning. This approach detects redundancy even when questions include varying empathetic elements or different phrasing, going beyond simple string comparison.

Some embodiments automatically analyze structured checklist data with severity ratings, apply category-specific thresholds, and generate prioritized screening recommendations with documented evidence. The system determines which assessments or screenings to recommend based on user-reported items and concerns, with the specific assessment types determined by the active assessment framework for the deployment domain. The embodiments described below may be implemented individually or in various combinations to provide flexible, safe, and effective AI-assisted intake capabilities across a plurality of deployment domains.

### System Architecture and Component Integration

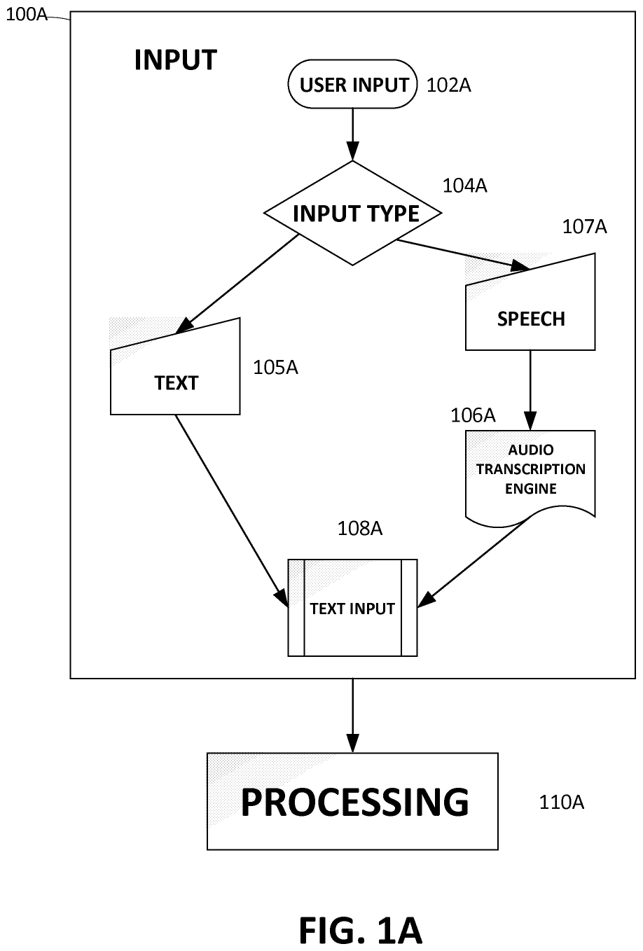

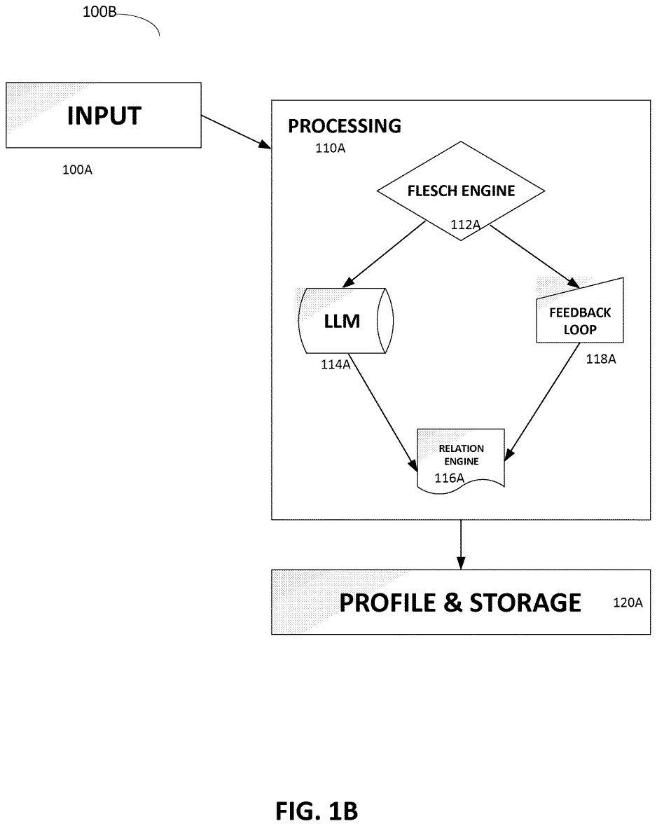

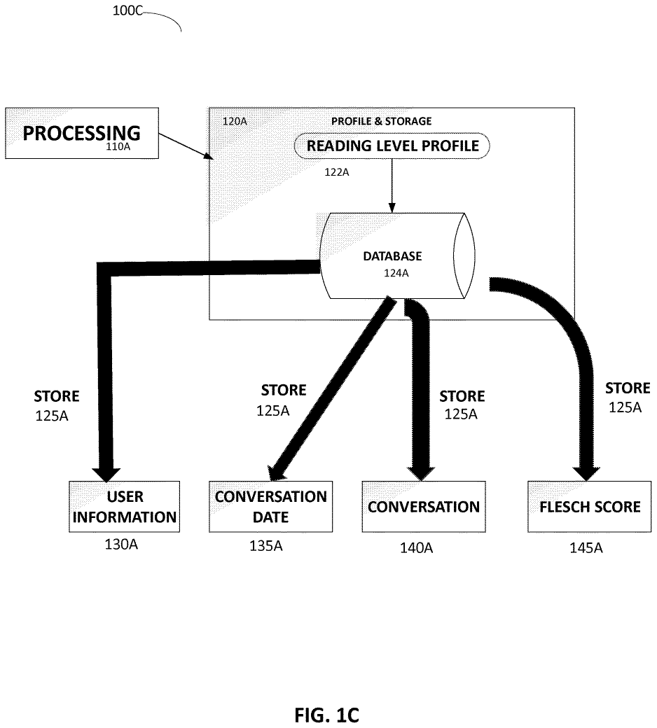

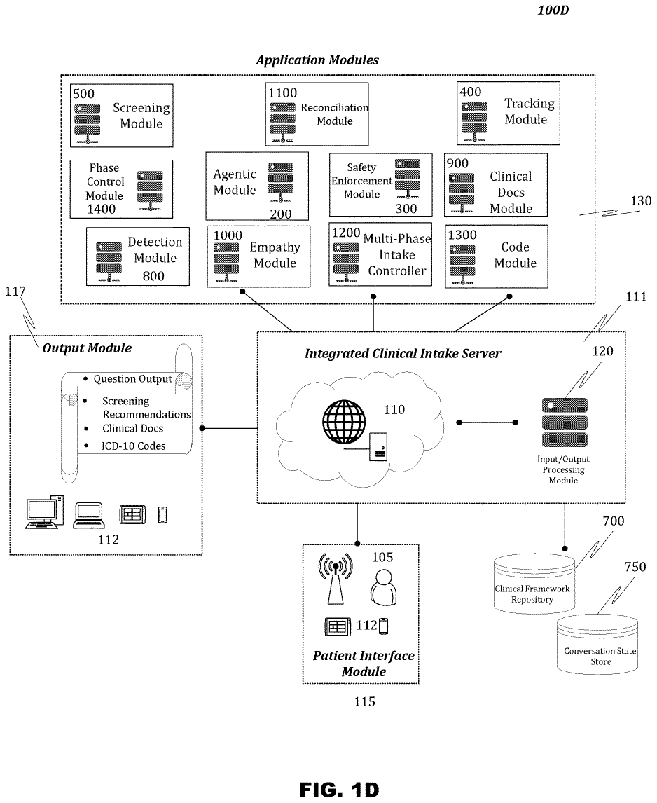

Referring to FIG. 1A, the integrated clinical intake system (100) may comprise: an Agentic Intake Engine (200); a Defense-in-Depth Safety Enforcement Layer (300); a Domain Tracking State Machine (400); a Screening Recommendation Engine (500); a Conversation State Store (750); a Clinical Framework Repository (700); a Proxy and Caregiver Detection System (800); an Automated Clinical Document Generation System (900); a Context-Aware Empathy Injection System (1000); a Real-Time Medication Reconciliation System (1100); a Multi-Phase Intake Controller (1200); an ICD-10 Code Suggestion System (1300); a Phase Control Module (1400); a Medical Advice Output Safety Filter (1500); a Red Flag Detection Engine (1600); a Stalled Coverage Detector (1700); an EHR Context Injection System (1800); and an Adaptive Readability and Linguistic Personalization Subsystem (1900).

Data may flow between these components as follows. The Multi-Phase Intake Controller (1200) may determine the current phase and may route user input accordingly. During initial interaction, the Virtual Agent Personality Module (1400) may present agent options and may capture personality selection. The Proxy and Caregiver Detection System (800) may scan for proxy indicators and may adjust question phrasing. User input may enter the Agentic Intake Engine (200) during the freeform AI phase. The engine may query the Defense-in-Depth Safety Enforcement Layer (300) for safety evaluation. The engine may update the Domain Tracking State Machine (400) with coverage information. The Context-Aware Empathy Injection System (1000) may ensure empathetic acknowledgments. The Adaptive Readability and Linguistic Personalization Subsystem (1900) may evaluate both user-provided text and AI-generated output using standardized readability metrics, may update user-specific linguistic preference profiles, and may constrain subsequent AI-generated output to match a target readability range. The subsystem (1900) may operate in parallel with the Empathy Injection System (1000) and prior to the Output Safety Filter (1500), such that linguistic personalization may occur without modifying mandatory safety enforcement, domain coverage tracking, or completion gate logic. The Output Safety Filter (1500) may scan and may remove domain-inappropriate advisory content from AI output. The Structured Data Reconciliation System (1100) may extract and may reconcile structured data items. Upon completion, the Screening Recommendation Engine (500) may generate recommendations, the Automated Document Generation System (900) may produce structured output documents, and the Domain Code Suggestion System (1300) may generate domain-appropriate classification suggestions. The External Records Context Injection System (1800) may provide user history from external systems through standard interoperability interfaces, such as FHIR R4 and HL7 v2 interfaces for healthcare deployments.

Regarding FIGS. 1A, 1B, and 1C, they illustrate a flowchart illustrating the overall process of the medical history collection system. Referring now to FIGS. 1A, 1B, and 1C, flowcharts illustrating the overall process of the medical history collection system may be seen. FIG. 1A, 100A depicts the input stage of the process. User input 102A may be received by the system. The input type may be determined at decision point 104A. If the input is text, it may proceed directly to text input 108A. If the input is speech, it may be processed by audio transcription engine 106A to convert the speech to text before proceeding to text input 108A. The text input may then be passed to the processing stage.

More specifically, referring to FIG. 1A, the integrated clinical intake system (100A) may comprise an input region (100A) that serves as the entry boundary for all patient-provided data. The input region (100A) may encompass the components responsible for receiving, classifying, and normalizing patient input prior to downstream processing. A user input block (102A) may serve as the entry point through which a patient may provide data to the system. The user input block (102A) may receive data originating from a patient interacting with the system through a client application, such as a web browser, mobile application, or desktop application. From the user input block (102A), a downward arrow may lead to an input type decision block (104A). The input type decision block (104A) may evaluate the nature of the incoming patient data to determine whether the data may be characterized as text-based or speech-based. The input type decision block (104A) may operate as a branching element that routes incoming data along one of two distinct processing pathways depending on the detected input modality. When the input type decision block (104A) determines that the incoming data may be text-based, the data may be routed along a left-branching pathway to a text block (105A). The text block (105A) may represent the direct text input pathway through which patient-typed responses may pass without requiring transcription or format conversion. When the input type decision block (104A) determines that the incoming data may be speech-based, the data may be routed along a right-branching pathway to a speech block (107A). The speech block (107A) may represent the audio input pathway through which patient-spoken responses may be received by the system.

From the speech block (107A), a downward arrow may lead to an audio transcription engine (106A). The audio transcription engine (106A) may receive the speech-based input from the speech block (107A) and may convert the audio data into a text representation. The audio transcription engine (106A) may utilize speech recognition functionality available through the device's native capabilities or through an external transcription service. Both the text block (105A) and the audio transcription engine block (106A) may direct their respective outputs toward a text input block (108A). The text input block (108A) may serve as the convergence point for all input modalities, receiving normalized text data regardless of whether the original patient input was provided through text entry or speech. The text input block (108A) may consolidate the two input pathways into a single unified text representation suitable for downstream processing. From the text input block (108A), a downward arrow may exit the input region boundary (100A) and may lead to a processing block (110A). The processing block (110A) may receive the normalized text input from the text input block (108A) and may perform further operations on the patient-provided data. The processing block (110A) may correspond to processing functionality described in connection with other figures, including readability evaluation, language model interaction, and clinical framework processing.

### Culturally Adaptive Virtual Agent System

Referring to FIG. 1B, the system may incorporate culturally adaptive virtual agents that dynamically adjust communication style, tone, and linguistic complexity based on patient preferences and demonstrated communication patterns. The Virtual Agent Personality Module (1400) may present patients with selectable AI personalities including encouraging/upbeat, professional/efficient, casual/approachable, warm/maternal, calm/gentle, and clinical/professional communication styles. Each personality may be layered on mandatory base guardrails that enforce safety protocols, clinical framework compliance, and empathy requirements.

Furthermore, FIG. 1B, 100B illustrates the processing stage of the system. The input received from the input stage 100A may enter the processing block 110A. Within the processing block, the input may be analyzed by Flesch engine 112A to determine the readability level. The input may also be processed by large language model (LLM) 114A to generate an initial response. The output of the Flesch engine 112A and LLM 114A may be passed to relation engine 116A, which may customize the response based on the determined readability level. A feedback loop 118A may allow for continuous refinement of the response. The customized response may then be passed to the profile and storage stage.

The system may implement a Docatar Relatable Response System (DRRS) comprising multi-layer cultural context embeddings that influence response generation based on user age, ethnicity, language preference, and inferred cultural context. The DRRS may identify cultural context through explicit user-provided attributes and implicit conversational signals, generating a cultural context vector (CCV) that activates hierarchical cultural embeddings across global, regional, national, and local levels.

The system may apply ethical and cultural content filtering through multiple stages including ethical compliance checks, cultural appropriateness classification, and sensitivity analysis. Cultural sensitivity evaluation metrics may include a Cultural Appropriateness Score (CAS), Diversity of Cultural References (DCR), Contextual Relevance Index (CRI), and Offensive Content Avoidance Rate (OCAR). These metrics may feed back into culturally diverse training data curation to improve model performance over time.

### Agentic Clinical Intake Engine: Technical Architecture

The system architecture implements an agentic approach to clinical intake, meaning the AI operates with a degree of autonomy while remaining constrained by deterministic safety guardrails. Referring to FIG. 1B, the Agentic Intake Engine (200) may comprise: a Framework Selector (202) using keyword matching with scoring; a Guardrails Generator (204) building clinical constraints including explicit domain coverage status from the state machine; a Unified Response Generator (206) making a single API call returning JSON with nextquestion, covereddomains, frameworkid, shouldcomplete, safetylevel, and clinicalnotes fields; an Output Module implementing least-recently-used (LRU) eviction with configurable maximum size (default: 128 entries, providing approximately 15-20% reduction in API call latency); and a Conversation Manager (210) maintaining conversation history and session state.

The Framework Selector (202) may analyze patient chief complaints and automatically select appropriate clinical questioning frameworks. For example, if a patient mentions chest pain, the system may select the OPQRST framework (Onset, Provocation, Quality, Radiation, Severity, Time). If a patient mentions feeling depressed, the system may select a mental health framework such as PHQ-9 for depression screening or GAD-7 for anxiety assessment. This selection may happen dynamically using keyword matching with scoring, allowing the system to adapt to each patient's unique situation.

The Unified Response Generator (206) may represent a key architectural innovation. Rather than making multiple separate calls to the AI language model for different purposes, this component may make a single API call that returns a comprehensive JSON response containing multiple fields: the next question to ask the patient, a list of which clinical domains have now been covered, the framework being used, a flag indicating whether the interview should be considered complete, an assessment of safety level, and preliminary clinical notes. This unified approach may reduce latency and ensure consistency across all determinations.

### Defense-In-Depth Safety Architecture

Referring to FIG. 1C, the system may implement a multi-layered safety enforcement architecture that operates on a “defense-in-depth” principle with multiple independent safety mechanisms. The Primary AI Safety Classifier (302) may use the language model's semantic understanding to analyze patient responses for safety concerns, distinguishing between figurative speech (“I'm dying to see that movie”) versus potential medical emergencies (“I feel like I'm dying”). The classifier may assign safety levels (LOW, MODERATE, HIGH, CRITICAL) and categorize the type of concern.

In some embodiments, regarding FIG. 1C, 100C the Processing module (110A) may implement real-time readability adjustment algorithms that modify AI-generated text on-the-fly before presentation to the patient. The module may receive draft question text from the Unified Response Generator (206), may compute the Flesch readability score of the draft text, and may compare the computed score against the target range retrieved from the Reading Level Profile (122A). If the draft text complexity may fall outside the target range, the Processing module (110A) may invoke text simplification or elaboration algorithms to adjust sentence structure, vocabulary selection, and grammatical complexity until the modified text may achieve a Flesch score within the target range. In further embodiments, the Database (124A) may support aggregate analytics across patient populations, enabling healthcare organizations to analyze readability patterns by demographic categories, clinical conditions, or geographic regions. The system may generate reports showing average Flesch scores for different patient cohorts, identifying populations that may benefit from simplified communication protocols or enhanced health literacy support services.

As such, FIG. 1C, 100C shows the profile and storage stage of the system. The output from the processing stage may enter the profile and storage block 120A. A reading level profile 122A may be updated based on the interaction. The database 124A may store (125A) various elements of the interaction, including user information, conversation date, conversation content, and Flesch score. This stored information may be used to personalize future interactions with the user.

Moreover, for FIG. 1C, the Profile & Storage module (120A) may update the Reading Level Profile (122A) after each interaction, incorporating new readability measurements into the patient's longitudinal profile. The updated profile may reflect the patient's evolving linguistic preferences and comprehension capabilities over time. The system may apply temporal weighting algorithms that give greater influence to recent interactions while preserving historical baseline measurements.

Regarding FIG. 1C, 100C, the Database (124A) may implement relational data structures that link user information (130A), conversation dates (135A), conversation content (140A), and Flesch readability scores (145A) through normalized database schemas. The database may support efficient retrieval of patient-specific readability profiles through indexed queries on user information fields. The database may maintain referential integrity constraints ensuring that each stored Flesch readability score may be associated with valid user information and conversation content records.

In some embodiments, the Database (124A) may store additional readability metrics beyond Flesch scores, including Flesch-Kincaid Grade Level, Gunning Fog Index, SMOG Index, Coleman-Liau Index, and Automated Readability Index. The system may compute multiple readability metrics for each patient response and may store the complete set of metrics in the database, enabling comparative analysis and selection of the most appropriate metric for specific patient populations or clinical contexts.

In further embodiments, the Profile & Storage module (120A) may implement privacy-preserving data storage techniques that encrypt conversation content (140A) and user information (130A) while maintaining the ability to retrieve and analyze readability scores. The system may apply encryption algorithms that protect patient confidentiality while enabling statistical analysis of readability patterns across patient populations.

The Reading Level Profile (122A) may comprise structured data fields including: average Flesch Reading Ease score computed across all stored interactions; standard deviation of Flesch scores indicating consistency of patient language complexity; minimum and maximum observed Flesch scores defining the patient's complexity range; preferred target Flesch score range for AI-generated output; interaction count indicating the number of stored conversations contributing to the profile; last updated timestamp indicating when the profile was most recently modified; and confidence score indicating the statistical reliability of the profile based on sample size and consistency.

The adaptive personalization enabled by the stored Flesch readability scores and user information may provide several technical advantages. First, the system may reduce patient confusion and improve comprehension by matching AI-generated language complexity to demonstrated patient capabilities. Second, the system may improve interview efficiency by avoiding overly simplified language for patients comfortable with medical terminology. Third, the system may enhance patient satisfaction by providing personalized communication that respects individual linguistic preferences and educational backgrounds.

The subsystem (100C) may operate in parallel with other system components including the Context-Aware Empathy Injection System (1000) and the Medical Advice Output Safety Filter (1500). The linguistic personalization may occur without modifying mandatory safety enforcement, domain coverage tracking, or completion gate logic. Safety-critical questions from the Always-Ask Registry (306) may not be simplified or altered, even for patients with low demonstrated reading levels, ensuring that critical clinical information may be collected with appropriate precision and clarity.

The data flow illustrated in FIG. 1C may represent a continuous feedback loop where patient language use may inform system output, which may in turn influence subsequent patient responses. This iterative adaptation may enable the system to converge on optimal communication complexity levels for each individual patient, improving both clinical data quality and patient experience throughout the intake process. The system depicted in FIGS. 1A-1C may allow for adaptive, personalized collection of medical history information from patients while adjusting the language and communication style based on the individual user's comprehension level and preferences.

The Secondary Deterministic Pattern Detector (304) may operate as a backup safety net using hardcoded pattern matching. This component may scan every patient response for specific keywords and phrases that indicate safety risks, including passive expressions of suicidal ideation that AI might miss, such as “I don't see the point anymore,” “nothing matters,” “everyone would be better off without me,” or “I just want to sleep and not wake up.” This detector may use deterministic logic that follows fixed rules independent of AI interpretation, covering enumerated safety categories including explicit and passive suicidal ideation, homicidal thoughts, child or elder abuse disclosures, domestic violence, sexual assault, medical emergencies, and acute substance intoxication or withdrawal.

The Always-Ask Registry (306) may maintain a list of questions that must be asked in certain contexts, regardless of what the AI thinks is appropriate. The Completion Gate Enforcer (308) may implement hard gates that prevent premature interview termination. When the AI indicates that the interview should be complete, the Completion Gate Enforcer may check whether all always-ask domains have been addressed and whether all required clinical domains have reached an acceptable coverage state.

The Safety Corroboration Module (310) may address AI hallucination of domain coverage by cross-checking the AI's claimed domain coverage against the actual conversation history and the state machine's records. If the AI claims a safety-critical domain was covered but the state machine shows it as NOT_STARTED or ASKED without a substantive answer, the corroboration module may flag the discrepancy and force the standard safety question to be asked explicitly.

### Domain Tracking and Framework Management

Referring to FIG. 1D, the Domain Tracking State Machine (400) may maintain a formal model of clinical completeness. Each clinical framework may define a set of domains that should be addressed, with the state machine tracking each domain's status and enforcing valid state transitions. The system may implement a two-pass coverage evaluation algorithm using keyword matching in the first pass to quickly identify obvious domain coverage, followed by AI semantic analysis in the second pass to identify more subtle coverage.

Furthermore, FIG. 1D may illustrate a system architecture diagram (100D) showing the Integrated Clinical Intake Server (111) as the central hub coordinating multiple application modules (130), patient interfaces (115), and data repositories. The Patient Interface Module (115) may capture patient input through various devices (112) and may communicate with the server (111) via wireless connectivity. A user or patient (105) may access the Patient Interface Module (115) through the various device (112). The Integrated Clinical Intake Server (111) may contain a cloud-based processing engine (110) and an Input/Output Processing Module (120) that routes data between components. The server may coordinate with multiple Application Modules (130) including the Agentic Module (200), Safety Enforcement Module (300), Tracking Module (400), Screening Module (500), Clinical Docs Module (900), Empathy Module (1000), Multi-Phase Intake Controller (1200), Code Module (1300), and Phase Control Module (1400). The Output Module (117) may present system-generated questions and clinical outputs to patients across multiple device types (desktop, laptop, tablet, smartphone, and various devices 112), displaying screening results, recommendations, clinical documentation, and ICD-10 codes. The Integrated Clinical Intake Server (111) may persist conversation state and retrieve clinical frameworks from backend data repositories (700 & 750). The Clinical Framework Repository (700) may store structured definitions of all available clinical frameworks. Each framework definition may include: applicability triggers, a list of domains with priority rankings, required versus optional domains, specific questions to ask for each domain, follow-up questions based on responses, skip logic, coverage indicators, clinical significance ratings, completion criteria, and always-ask domains that cannot be skipped. The Conversation State Store (750) may store conversation state date and relevant contextual information.

### Automated Clinical Documentation Generation

The Automated Clinical Document Generation (900) component may transform the conversational intake into professional clinical documentation. The system may generate multiple documentation formats depending on the clinical context. For primary care encounters, it may produce SOAP notes (Subjective, Objective, Assessment, Plan). For behavioral health encounters, it may generate comprehensive psychiatric evaluation reports with up to 21 standardized sections. The documentation generation may include clinical language transformation, the system may convert casual patient language into appropriate medical terminology while preserving the patient's voice where clinically relevant. For example, if a patient says “my stomach has been really messed up,” the documentation might read “Patient reports gastrointestinal distress” while noting the patient's characterization. The system may implement source attribution, indicating which statements came directly from the patient versus which were inferred or observed.

### Medication Reconciliation and EHR Integration

The Medication Reconciliation (1100) component may extract structured medication information from natural conversation. When a patient mentions medications, the system may parse out the medication name, dosage, frequency, what condition it treats, who prescribed it, and current status. The system may then reconcile this information against the patient's medication list retrieved from the EHR, flagging discrepancies and generating follow-up probes to gather missing details.

The EHR Context Injection (1800) component may retrieve relevant patient information from the EHR at the start of the intake session. The system may support FHIR R4 and HL7 v2 messaging to communicate with diverse EHR systems. Critically, the system may implement a staged approval workflow where all AI-generated updates are staged for provider review rather than being automatically committed to the permanent medical record.

### Adaptive Readability and Linguistic Personalization

The Adaptive Readability & Linguistic Personalization (1900) component may tailor the system's communication style to match the patient's preferences and comprehension level. The system may estimate the readability level of its own outputs using metrics such as Flesch Reading Ease or Flesch-Kincaid Grade Level. It may also learn from patient feedback, adjusting complexity downward if a patient indicates confusion or using more technical language if a patient is clearly comfortable with medical terminology. The system may store user linguistic preference profiles that capture average readability scores, preferred ranges, satisfaction indicators, personality preferences, and lexical and sentence-length preferences. Importantly, this personalization may operate within constraints, the system cannot simplify or alter safety questions, mandatory clinical content, or domain coverage requirements.

### Multi-Phase Intake Orchestration

The Multi-Phase Intake Controller (1200) may manage the overall structure of the intake process through multiple phases: initial greeting and consent, demographic information collection, chief complaint identification, structured problem checklist, conversational history-taking guided by selected frameworks, standardized assessment administration, medication reconciliation, review and confirmation, and closeout with summary. The controller may enforce question budgets, manage context bridging, and ensure that mandatory phases cannot be skipped.

### Operational Flow and System Integration

In a typical operational flow, a patient may receive an invitation link via SMS or email and open the intake session on their device. The system may present consent information and capture the patient's agreement. The patient may select their preferred communication style and begin the conversation by describing their chief complaint. The Framework Selector may analyze this input and select an appropriate clinical framework. As the patient responds, each answer may flow through the safety layers, update the domain tracking state machine, and inform the next question generation.

Throughout the conversation, the Conversation Manager may maintain context, the Redundancy Detection may prevent repeated questions, and the Empathy Injection may ensure appropriate acknowledgment of patient concerns. When the AI determines the interview could be complete, the Completion Gate Enforcer may verify that all always-ask domains have been addressed. Only when all mandatory requirements are satisfied may the system allow the interview to conclude.

Upon completion, the Documentation Generator may produce clinical notes, the Screening Recommendation Engine may compile suggested assessments with evidence, the ICD-10 Suggestion System may propose diagnosis codes, and all medication reconciliation findings may be compiled. These outputs may be staged for provider review. A clinician may log into the provider portal, review the intake materials including any safety flags, approve accurate items, edit as needed, and commit the approved content to the EHR.

This architecture may enable the system to conduct clinical intake interviews that are simultaneously conversational and natural, clinically rigorous and complete, and operationally efficient, while maintaining the fundamental principle that licensed clinical professionals retain final authority over medical records and patient care decisions. The embodiments described may be implemented individually or in various combinations to provide flexible, safe, and effective AI-assisted clinical intake capabilities with culturally adaptive communication, defense-in-depth safety enforcement, and comprehensive clinical documentation generation.

### Domain-Agnostic Architecture and Deployment Scenarios

The agentic intake engine architecture described herein may be deployed across a plurality of domains without modification to the core engine components. The domain tracking state machine, defense-in-depth safety enforcement layer, hard completion gates, guardrails injection mechanism, empathy injection subsystem, readability personalization subsystem, proxy detection system, multi-phase intake controller, and structured document generation subsystem may each operate independently of the specific subject matter domain in which the system is deployed. The assessment framework repository may store a plurality of frameworks, each defining a set of domains with coverage indicators, priority values, and required flags appropriate to a specific deployment context. The agentic intake engine may select among stored frameworks based on keyword analysis of the initial user input, enabling a single deployed system to serve multiple intake contexts without architectural changes.

In a healthcare deployment scenario, the system may select a healthcare assessment framework comprising domains such as chief complaint, medical history, current medications, allergies, social history, family history, review of systems, and safety screening. The structured output document may comprise a clinical note formatted for review by a licensed healthcare provider. The external records context injection subsystem may authenticate with electronic health record systems via FHIR R4 interfaces to retrieve prior encounter data. Safety categories enforced by the defense-in-depth layer may include suicidal ideation, homicidal ideation, substance abuse crisis, and domestic violence disclosure. The domain code suggestion subsystem may generate ICD-10 diagnostic code suggestions based on conversation analysis.

In a human resources onboarding deployment scenario, the system may select an HR onboarding assessment framework comprising domains such as employment eligibility, prior employment history, compensation preferences, benefits enrollment, policy acknowledgment, equipment provisioning, and emergency contact collection. The structured output document may comprise an onboarding intake record formatted for review by an HR representative. Safety categories enforced by the defense-in-depth layer may include workplace harassment disclosure, hostile work environment reporting, and occupational safety concerns. The domain code suggestion subsystem may generate role classification or job code suggestions based on conversation analysis.

In a legal intake deployment scenario, the system may select a legal intake assessment framework comprising domains such as matter type identification, adverse party identification, conflict of interest screening, statute of limitations assessment, prior representation history, and fee arrangement disclosure. The structured output document may comprise a matter intake summary formatted for review by a licensed attorney. Safety categories enforced by the defense-in-depth layer may include imminent physical harm disclosure, domestic violence reporting, and child welfare concerns requiring mandatory reporting. The domain code suggestion subsystem may generate matter type classification or practice area routing suggestions based on conversation analysis.

In a financial compliance deployment scenario, the system may select a financial compliance assessment framework comprising domains such as customer identification, beneficial ownership disclosure, source of funds verification, politically exposed person screening, sanctions screening, and risk tolerance assessment. The structured output document may comprise a regulatory disclosure report formatted for review by a compliance officer. Safety categories enforced by the defense-in-depth layer may include fraud indicators, money laundering red flags, and sanctions match disclosures requiring escalation. The domain code suggestion subsystem may generate regulatory classification or risk rating suggestions based on conversation analysis.

In each deployment scenario described above, the domain tracking state machine may track coverage of each domain through discrete states comprising NOT_STARTED, ASKED, ANSWERED, INFERRED, SKIPPED, and BLOCKED, with enforced state transitions. Hard completion gates may prevent session termination until all domains designated as required in the active assessment framework have been addressed, regardless of AI-generated completion signals, user requests, or configuration changes. The defense-in-depth safety enforcement layer may apply both primary AI semantic classification and secondary deterministic pattern matching against keyword libraries organized across a plurality of safety categories specific to the deployment domain. The readability personalization subsystem may adapt AI-generated output to match each user's demonstrated linguistic comprehension level across all deployment scenarios. The empathy injection subsystem may inject context-appropriate empathetic prefixes when empathy markers are absent from AI-generated output across all deployment scenarios.

### Clinical Utilization Scenarios and Professional Workflows

### Primary Care Physician Workflow

Dr. Sarah Chen, a family medicine physician, may utilize the system to streamline her busy primary care practice. When a patient schedules an appointment for “stomach problems,” the system may automatically send an intake invitation 24 hours before the visit. The patient may complete the intake from home, selecting the “Professional/Efficient” virtual agent personality. The Framework Selector may identify the chief complaint and automatically deploy the OPQRST framework (Onset, Provocation, Quality, Radiation, Severity, Time) along with a comprehensive gastrointestinal assessment framework.

During the intake, the patient may describe intermittent abdominal pain that worsens after eating. The Domain Tracking State Machine may systematically address pain characteristics, associated symptoms, dietary factors, bowel habits, and medication history. The Defense-in-Depth Safety Layer may scan for red flag symptoms such as “severe pain,” “blood in stool,” or “unintentional weight loss,” automatically escalating the urgency level if detected.

When Dr. Chen reviews the intake before the appointment, she may receive a structured SOAP note highlighting key findings: the patient reports postprandial epigastric pain, denies alarm symptoms, has a family history of gallbladder disease, and takes ibuprofen regularly. The Screening Recommendation Engine may suggest H. pylori testing and abdominal ultrasound based on symptom patterns. The ICD-10 Code Suggestion System may propose K30 (functional dyspepsia) as a preliminary diagnosis code. This preparation may allow Dr. Chen to focus the in-person visit on physical examination and clinical decision-making rather than repetitive history-taking.

### Emergency Department Nurse Practitioner Scenario

Jennifer Martinez, NP, may work in a busy emergency department where rapid triage and assessment are critical. When a patient presents with chest pain, the triage nurse may initiate the intake system on a tablet while obtaining vital signs. The system may immediately deploy the chest pain framework with enhanced safety protocols, given the emergency setting. The patient may describe crushing chest pain radiating to the left arm that started 2 hours ago. The Primary AI Safety Classifier may immediately flag this as CRITICAL severity, while the Secondary Deterministic Pattern Detector may identify keywords like “crushing,” “radiating,” and “chest pain” in its cardiac emergency library. The Always-Ask Registry may enforce mandatory questions about cardiac risk factors, current medications, and allergies, regardless of time constraints.

The Completion Gate Enforcer may prevent the intake from concluding until all cardiac-specific domains are addressed: pain characteristics, associated symptoms, cardiac history, risk factors, and current medications. When Jennifer reviews the intake, she may receive an automatically generated emergency assessment note with highlighted red flags, medication reconciliation showing the patient takes aspirin and metoprolol, and screening recommendations for immediate EKG and cardiac enzymes. The system may also generate a structured handoff report for the attending physician, ensuring critical information is not lost during care transitions.

### Behavioral Health Psychiatrist Implementation

Dr. Michael Rodriguez, a psychiatrist, may utilize the system for comprehensive psychiatric evaluations. When a patient is referred for depression screening, the system may deploy specialized mental health frameworks including PHQ-9, GAD-7, and comprehensive psychiatric history modules. The patient may select the “Warm/Maternal” virtual agent personality to create a more comfortable environment for sensitive disclosures.

During the intake, the patient may describe feeling “hopeless” and having “no energy for weeks.” The Defense-in-Depth Safety Layer may immediately activate enhanced suicide risk protocols. The Primary AI Safety Classifier may detect concerning language patterns, while the Secondary Deterministic Pattern Detector may flag phrases like “no point in trying” and “nothing matters.” The Always-Ask Registry may enforce mandatory suicide risk assessment questions that cannot be bypassed.

The system may systematically address psychiatric history, substance use, trauma history, family psychiatric history, current medications, and psychosocial stressors. The Context-Aware Empathy Injection System may provide appropriate acknowledgments like “That sounds really difficult” while maintaining clinical focus. When Dr. Rodriguez reviews the intake, he may receive a comprehensive psychiatric evaluation report with structured sections for chief complaint, history of present illness, psychiatric history, substance use history, family history, mental status examination findings, and risk assessment. The system may highlight safety concerns and suggest immediate interventions if high-risk factors are detected.

### Pediatric Nurse Scenario

Lisa Thompson, RN, may work in a pediatric clinic where the Proxy and Caregiver Detection System becomes essential. When a mother brings her 8-year-old child for a wellness visit, the system may automatically detect the proxy scenario through phrases like “my son has been” or “he seems to be.” The system may adjust all subsequent questions to use third-person references: “Has your child experienced any fever?” rather than “Have you experienced any fever?”

The Virtual Agent Personality Module may present child-friendly communication options, and the mother may select “Casual/Approachable” to maintain a comfortable atmosphere. The system may deploy pediatric-specific frameworks addressing growth and development, immunization status, school performance, and behavioral concerns. The Adaptive Readability and Linguistic Personalization Subsystem may adjust language complexity to match the caregiver's demonstrated comprehension level.

When addressing sensitive topics like behavioral concerns or family stress, the Defense-in-Depth Safety Layer may activate child protection protocols, scanning for indicators of abuse or neglect. The Always-Ask Registry may enforce mandatory questions about safety at home, school performance, and social interactions. Lisa may receive a comprehensive pediatric assessment with age-appropriate developmental milestones, immunization recommendations, and any flagged safety concerns requiring immediate attention.

### Clinical Pharmacist Medication Reconciliation

PharmD candidate Robert Kim may utilize the system's Real-Time Medication Reconciliation capabilities during hospital admission processes. When a patient is admitted with multiple chronic conditions, the system may automatically extract medication information from natural conversation and reconcile it against the patient's medication list from the EHR.

The patient may mention taking “a little white pill for my heart” and “something for diabetes twice a day.” The medication reconciliation system may use pattern matching to identify potential medications: the heart medication may be flagged as potentially metoprolol or amlodipine based on description, while the diabetes medication may be identified as metformin based on dosing frequency. The system may generate targeted follow-up probes: “Can you tell me the exact name of your heart medication?” or “Do you remember the strength of your diabetes medication?”

The system may classify each medication as confirmed, new, discontinued, changed, or needs_followup, providing Robert with a structured medication reconciliation report. Discrepancies between patient-reported medications and EHR records may be highlighted for clinical attention. The system may also flag potential drug interactions, duplicate therapies, or missing medications based on the patient's condition profile.

### Psychology Practice Integration

Dr. Amanda Foster, a clinical psychologist, may implement the system for comprehensive psychological assessments. When a patient seeks therapy for anxiety, the system may deploy specialized psychological frameworks including GAD-7, Beck Anxiety Inventory, and trauma screening tools. The patient may select the “Calm/Gentle” virtual agent personality to create a therapeutic environment.

The system may systematically explore anxiety symptoms, triggers, coping mechanisms, trauma history, and psychosocial stressors. The Context-Aware Empathy Injection System may provide therapeutic acknowledgments while maintaining assessment structure. The Defense-in-Depth Safety Layer may monitor for self-harm indicators or severe psychological distress requiring immediate intervention.

Dr. Foster may receive a comprehensive psychological assessment report with standardized scoring for administered instruments, detailed symptom analysis, and treatment recommendations. The system may suggest evidence-based interventions based on symptom patterns and patient preferences, supporting treatment planning and clinical decision-making.

### Specialty Clinic Workflow—Cardiology

Dr. James Park, a cardiologist, may utilize the system for specialized cardiac consultations. When a patient is referred for chest pain evaluation, the system may deploy comprehensive cardiac assessment frameworks including detailed symptom characterization, cardiac risk factor analysis, and functional capacity evaluation. The system may systematically address chest pain characteristics using the OPQRST framework, explore cardiac risk factors including family history, smoking, diabetes, and hypertension, and assess functional limitations through standardized questionnaires. The EHR Context Injection System may retrieve prior cardiac studies, medications, and procedures to inform questioning.

Dr. Park may receive a structured cardiac consultation report with risk stratification, functional assessment, and recommendations for diagnostic testing. The system may suggest appropriate cardiac studies based on symptom patterns and risk factors, supporting efficient and targeted clinical evaluation.

### Nurse Manager Quality Assurance

Sarah Williams, RN, MSN, may serve as a nurse manager utilizing the system's comprehensive audit and quality assurance capabilities. The system may provide detailed analytics on intake completion rates, safety flag frequencies, and clinical documentation quality across her unit. The system may generate reports showing which providers consistently achieve complete domain coverage, which safety protocols are most frequently triggered, and which patient populations require additional support. Sarah may use this data to identify training needs, optimize workflows, and ensure consistent quality of care across her nursing staff.

The staged approval workflow may allow Sarah to review and approve intake documentation before it enters the permanent medical record, ensuring quality control and regulatory compliance. The system may maintain comprehensive audit trails documenting all clinical decisions and provider actions for quality assurance and risk management purposes.

### Telemedicine Integration Scenario

Dr. Patricia Lee may conduct telemedicine visits enhanced by the intake system. Patients may complete comprehensive intake sessions before their virtual appointments, allowing Dr. Lee to focus video time on clinical decision-making rather than information gathering. The system may provide structured intake documentation that integrates seamlessly with telemedicine platforms, highlighting key clinical findings and safety concerns for immediate attention. The Adaptive Readability and Linguistic Personalization Subsystem may ensure that patients with varying technological comfort levels can successfully complete the intake process.

The Multi-Phase Intake Controller may coordinate with telemedicine scheduling systems, automatically triggering intake invitations at appropriate intervals before appointments and ensuring that all necessary information is collected before the virtual encounter begins.

### Specialized High-Acuity and Vulnerable Population Scenarios

### Severe Mental Health Crisis Intervention

Dr. Rachel Martinez, a crisis intervention psychiatrist, may utilize the system in an inpatient psychiatric unit where patients present with severe mental health emergencies. When a patient is admitted following a suicide attempt, the system may immediately activate maximum safety protocols with enhanced monitoring capabilities. The Defense-in-Depth Safety Layer may implement continuous safety scanning with zero tolerance thresholds for any concerning language.

The system may deploy specialized frameworks for acute psychiatric assessment including Columbia Suicide Severity Rating Scale, Beck Scale for Suicide Ideation, and comprehensive risk factor evaluation. The Always-Ask Registry may enforce mandatory questions about current suicidal ideation, access to means, protective factors, and support systems. The Virtual Agent Personality Module may present only “Clinical/Professional” and “Calm/Gentle” options to maintain therapeutic boundaries while providing emotional support. The Completion Gate Enforcer may implement absolute requirements that prevent any interview conclusion until comprehensive suicide risk assessment is completed, including detailed safety planning and crisis resource identification. Dr. Martinez may receive real-time alerts for any detected safety concerns, with automatic escalation protocols triggering immediate clinical intervention when high-risk indicators are identified.

### Substance Abuse Treatment Center Implementation

At a residential substance abuse treatment facility, counselor Mark Thompson may utilize the system for comprehensive addiction assessments. When patients enter detoxification programs, the system may deploy specialized substance use frameworks including AUDIT (Alcohol Use Disorders Identification Test), DAST (Drug Abuse Screening Test), and comprehensive withdrawal assessment protocols.

The system may systematically address substance use history, withdrawal symptoms, medical complications, psychiatric comorbidities, and social support systems. The Defense-in-Depth Safety Layer may monitor for medical emergency indicators including severe withdrawal symptoms, delirium tremens risk factors, or suicidal ideation related to substance use. The Secondary Deterministic Pattern Detector may scan for phrases indicating medical instability: “shaking uncontrollably,” “seeing things,” “chest pain,” or “can't breathe.”

The Real-Time Medication Reconciliation System may be particularly critical in this setting, identifying potential drug interactions between prescribed medications and substances of abuse. The system may generate comprehensive addiction treatment plans with evidence-based intervention recommendations, relapse prevention strategies, and medical monitoring requirements.

### Trauma-Informed Care in Emergency Settings

In a Level I trauma center, Dr. Jennifer Kim may utilize the system for patients presenting with acute trauma histories. The system may implement trauma-informed communication protocols that recognize the potential for re-traumatization during clinical interviews. The Virtual Agent Personality Module may emphasize “Warm/Maternal” and “Calm/Gentle” options while maintaining clinical efficiency. The system may deploy specialized trauma assessment frameworks including PC-PTSD-5 (Primary Care PTSD Screen), ACE (Adverse Childhood Experiences) questionnaire, and comprehensive trauma history evaluation. The Context-Aware Empathy Injection System may provide enhanced emotional validation while maintaining clinical boundaries. The Defense-in-Depth Safety Layer may monitor for dissociation indicators, panic responses, or re-traumatization signs. The Proxy and Caregiver Detection System may be enhanced to identify potential perpetrators accompanying victims, automatically adjusting questioning to ensure patient safety. The system may provide discrete safety planning resources and automatically connect patients with trauma specialists and victim advocacy services.

### Military Battlefield Triage Integration

Combat medic Sergeant Lisa Rodriguez may utilize a ruggedized version of the system in forward operating base medical facilities. The system may implement rapid triage protocols with streamlined frameworks focused on immediate life-threatening conditions. The Multi-Phase Intake Controller may prioritize critical assessment domains while maintaining comprehensive documentation for medical evacuation purposes.

The system may deploy military-specific assessment frameworks including blast injury protocols, combat stress evaluation, and traumatic brain injury screening. The Defense-in-Depth Safety Layer may monitor for indicators of severe psychological trauma, including combat-related PTSD symptoms and suicidal ideation related to combat experiences.

The Adaptive Readability and Linguistic Personalization Subsystem may adjust for combat stress-related cognitive impairment, simplifying language and repeating critical questions as needed. The system may generate rapid medical evacuation documentation with standardized military medical terminology and priority classifications.

### Radiation Oncology Treatment Centers

Dr. Sarah Chen may utilize the system in a radiation oncology department where patients undergo complex cancer treatments. The system may deploy specialized oncology frameworks addressing treatment side effects, symptom management, and quality of life assessment. The Defense-in-Depth Safety Layer may monitor for severe treatment complications requiring immediate medical intervention.

The system may systematically assess radiation-related side effects including skin reactions, fatigue, nausea, and organ-specific toxicities. The Real-Time Medication Reconciliation System may track complex medication regimens including chemotherapy agents, supportive care medications, and symptom management drugs. The system may identify potential drug interactions and contraindications specific to radiation therapy protocols. The Context-Aware Empathy Injection System may provide enhanced emotional support for patients facing life-threatening diagnoses while maintaining clinical focus on treatment monitoring and safety assessment. The system may generate comprehensive treatment monitoring reports with standardized toxicity grading and intervention recommendations.

### Pediatric Oncology and End-of-Life Care

In a children's hospital oncology unit, Dr. Michael Foster may utilize the system for pediatric cancer patients and their families. The Proxy and Caregiver Detection System may identify complex family dynamics involving multiple caregivers, medical decision-makers, and support persons. The system may adjust communication to address both pediatric patients and their caregivers simultaneously.

The system may deploy age-appropriate assessment frameworks addressing treatment tolerance, developmental concerns, and family coping mechanisms. The Defense-in-Depth Safety Layer may monitor for indicators of severe distress in both patients and caregivers, including anticipatory grief, family conflict, and caregiver burnout.

The Virtual Agent Personality Module may present child-friendly communication options while maintaining sensitivity to serious medical conditions. The system may generate comprehensive family-centered care plans addressing medical treatment, psychosocial support, and end-of-life planning when appropriate.

### Hospice and Palliative Care Settings

Hospice nurse practitioner Dr. Amanda Wilson may utilize the system for comprehensive end-of-life care assessment. The system may deploy specialized palliative care frameworks addressing pain management, symptom control, spiritual concerns, and family support needs. The Defense-in-Depth Safety Layer may monitor for inadequate pain control, severe distress, or family crisis situations. The system may systematically assess pain levels using validated scales, evaluate symptom burden across multiple domains, and address advance directive preferences. The Context-Aware Empathy Injection System may provide enhanced emotional support while maintaining dignity and respect for end-of-life preferences.

The Real-Time Medication Reconciliation System may track complex pain management regimens including opioid medications, adjuvant therapies, and comfort care medications. The system may generate comprehensive comfort care plans with detailed symptom management protocols and family support resources.

### Geriatric and Memory Care Facilities

In a specialized memory care unit, geriatrician Dr. Robert Martinez may utilize the system for patients with cognitive impairment. The Adaptive Readability and Linguistic Personalization Subsystem may adjust communication complexity for varying levels of cognitive function, implementing simplified language and repetitive questioning as needed. The system may deploy specialized geriatric assessment frameworks including Mini-Mental State Examination, Geriatric Depression Scale, and comprehensive functional assessment tools. The Defense-in-Depth Safety Layer may monitor for indicators of elder abuse, neglect, or severe cognitive decline requiring immediate intervention. The Proxy and Caregiver Detection System may identify complex care relationships involving family members, professional caregivers, and legal guardians. The system may adjust questioning to gather information from multiple sources while maintaining patient dignity and autonomy when possible.

### Developmental Disability and Autism Spectrum Support

In a developmental disability clinic, Dr. Jennifer Park may utilize the system for patients with intellectual disabilities and autism spectrum disorders. The Adaptive Readability and Linguistic Personalization Subsystem may implement specialized communication protocols including visual supports, simplified language, and extended response times. The system may deploy developmental disability-specific frameworks addressing adaptive functioning, behavioral concerns, and support needs. The Virtual Agent Personality Module may present communication options optimized for neurodivergent individuals, including structured, predictable interaction patterns.

The Defense-in-Depth Safety Layer may monitor for indicators of abuse or exploitation, which individuals with developmental disabilities may be at increased risk for experiencing. The system may implement enhanced protection protocols and automatic connection with disability advocacy services when concerns are identified.

### Pediatric Foster Care and Child Protective Services

Social worker Maria Gonzalez may utilize the system in child protective services investigations and foster care placements. The system may implement specialized child protection frameworks addressing safety concerns, trauma history, and placement needs. The Defense-in-Depth Safety Layer may monitor for indicators of abuse, neglect, or unsafe living conditions. The Proxy and Caregiver Detection System may identify complex family structures involving biological parents, foster parents, caseworkers, and legal guardians. The system may adjust questioning to gather information from multiple sources while maintaining child safety and confidentiality.

The system may generate comprehensive child welfare assessments with standardized risk evaluation tools, safety planning protocols, and service coordination recommendations. The Always-Ask Registry may enforce mandatory questions about safety, basic needs, and protective factors that cannot be bypassed regardless of time constraints or other pressures.

### Emergency Department Psychiatric Crisis

In a busy emergency department, psychiatric emergency nurse Tom Anderson may utilize the system for patients presenting with acute mental health crises. The system may implement rapid psychiatric triage protocols with streamlined safety assessment frameworks. The Defense-in-Depth Safety Layer may provide continuous monitoring for self-harm risk, violence potential, and psychiatric emergency indicators.

The system may deploy emergency psychiatric frameworks including rapid suicide risk assessment, violence risk evaluation, and psychiatric emergency protocols. The Always-Ask Registry may enforce mandatory questions about immediate safety, current symptoms, and crisis precipitants that must be addressed before any disposition decisions.

The Multi-Phase Intake Controller may prioritize critical safety domains while maintaining comprehensive documentation for psychiatric consultation and disposition planning. The system may generate rapid psychiatric emergency assessments with clear recommendations for immediate interventions, safety precautions, and follow-up care.

These specialized scenarios may demonstrate the system's adaptability to high-acuity clinical situations and vulnerable populations requiring enhanced safety protocols, specialized communication approaches, and comprehensive documentation standards. The system may maintain consistent clinical rigor while adapting to the unique needs of diverse patient populations and clinical settings.

These diverse utilization scenarios may demonstrate the system's flexibility and comprehensive applicability across healthcare settings, professional roles, and clinical specialties. The system may adapt to specific workflow requirements while maintaining consistent safety protocols, clinical rigor, and documentation standards across all implementations.

### Multi-Phase Intake Architecture and System Integration

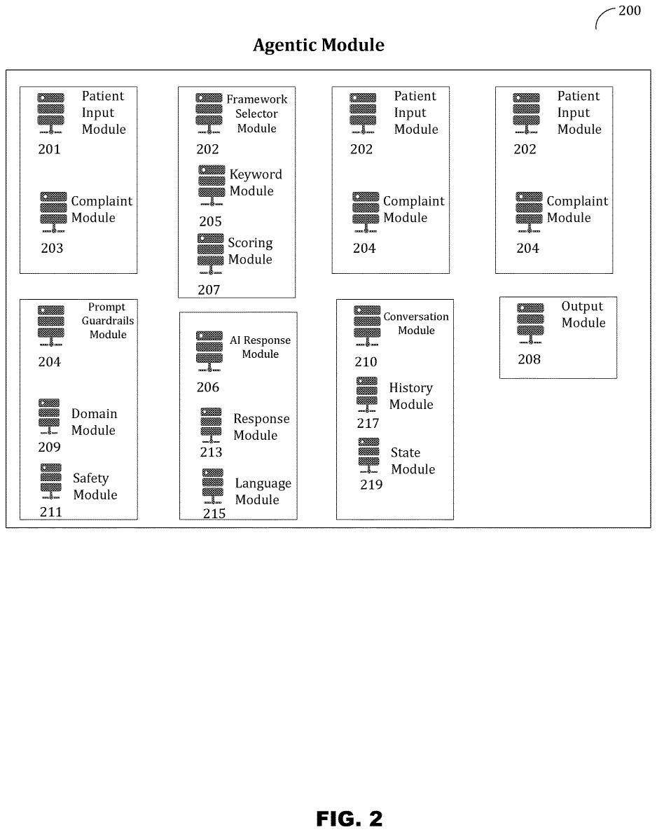

Referring to FIG. 2, the Multi-Phase Intake Controller (1200) may orchestrate a sophisticated sequence diagram of system interactions across multiple phases. The controller may manage the overall structure of the intake process through multiple phases: initial greeting and consent, demographic information collection, chief complaint identification, structured problem checklist, conversational history-taking guided by selected frameworks, standardized assessment administration, medication reconciliation, review and confirmation, and closeout with summary. The controller may enforce question budgets, manage context bridging, and ensure that mandatory phases cannot be skipped.

In one or more embodiments, temporal interactions between the patient interface, the Multi-Phase Intake Controller (1200), the Agentic Intake Engine (200), the Defense-in-Depth Safety Enforcement Layer (300), the Domain Tracking State Machine (400), and the Clinical Framework Repository (700). Each conversation turn may trigger a cascade of system interactions: patient input may be received and immediately routed through safety classification layers, domain coverage may be updated in real-time, framework-specific guardrails may be dynamically generated, and the next contextual question may be produced through unified API calls.

Furthermore, FIG. 2 may illustrate the Agentic Module (200) architecture showing the modular components that coordinate patient interaction, clinical framework selection, prompt construction, AI response generation, and conversation state management. The Patient Input Module (201) may receive and process patient responses. The Complaint Module (203) may analyze the patient's chief complaint to determine clinical context. The Prompt Guardrails Module (204) may enforce safety constraints and domain coverage requirements on AI prompts. The Keyword Module (205) may perform rapid keyword-based analysis to identify clinical terms and domain indicators in patient responses. The AI Response Module (206) may coordinate the generation of contextually appropriate follow-up questions using the language model. The Scoring Module (207) may evaluate response quality and domain coverage completeness. The Output Module (208) may format and deliver system-generated questions to the patient interface. The Domain Module (209) may track which clinical domains have been addressed and which remain uncovered. The Conversation Module (210) may maintain the complete interaction history throughout the intake session. The Safety Module (211) may enforce always-ask requirements and safety-critical question protocols. The Response Module (213) may validate AI-generated responses for clinical appropriateness and completeness. The Language Module (215) may adapt linguistic complexity to match patient reading level and comprehension capabilities. The History Module (217) may retrieve prior conversation turns to provide context for prompt construction. The State Module (219) may persist and retrieve conversation state across system components, ensuring continuity throughout the multi-turn clinical intake interview.

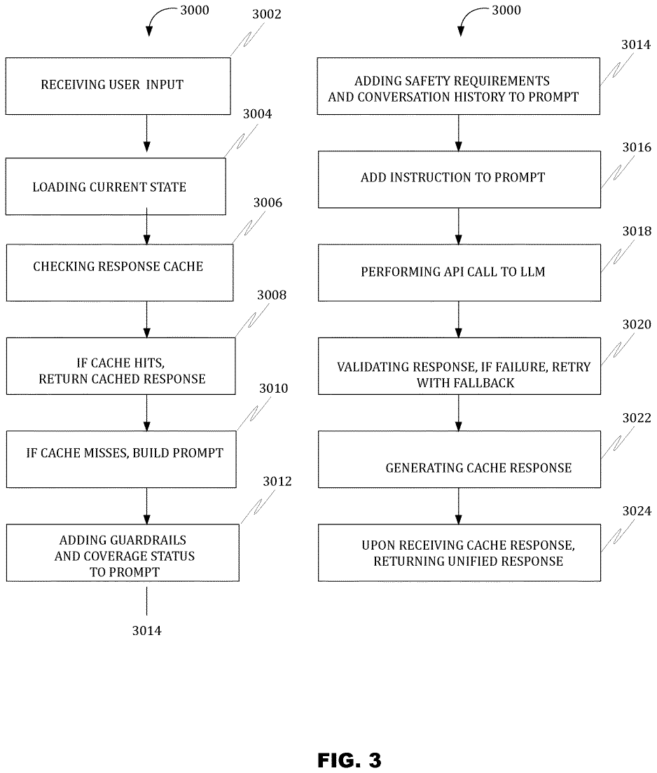

FIG. 3 may illustrate a method flow chart (3000) showing the two-phase process for generating unified AI responses during clinical intake interviews. The method may begin with receiving patient input (3002), followed by loading current state (3004) to retrieve conversation history and domain tracking status. The system may check a response cache (3006) to determine if an identical or similar patient input has been previously processed. If a cache hit occurs, the system may return the cached response (3008), improving response time and computational efficiency. If the cache misses, the system may build a prompt (3010) by assembling patient input, conversation context, and clinical framework requirements. The system may add guardrails and coverage status to the prompt (3012) to constrain AI behavior and communicate which clinical domains remain uncovered. The system may add safety requirements and conversation history to the prompt (3014) to enforce always-ask protocols and provide conversational continuity. The system may add instructions to the prompt (3016) specifying response format, linguistic complexity targets, and clinical documentation requirements. The system may perform an API call to the LLM (3018) to generate a candidate response based on the constructed prompt. The system may validate the response (3020), checking for safety violations, domain coverage progress, and clinical appropriateness; if validation fails, the system may retry with fallback prompts or alternative generation strategies. The system may generate a cache response (3022) by storing the validated AI output with associated metadata for future retrieval. Upon receiving the cache response, the system may return a unified response (3024) to the patient interface, completing the request-response cycle and enabling the next conversational turn in the clinical intake interview.

### Technological Advantages of Multi-Phase Architecture

The multi-phase approach may provide several technological advantages over traditional linear intake systems. First, context bridging may enable seamless data flow between phases by serializing structured data from prior phases into AI prompts, ensuring that demographic information, chief complaints, and problem checklist responses inform subsequent conversational questioning. This may eliminate redundant questioning and improve clinical efficiency.

In one or more embodiments, the system may implement adaptive phase sequencing, where the order and content of phases may be dynamically adjusted based on patient responses. For example, if a patient indicates acute distress during the chief complaint phase, the system may immediately escalate to safety screening phases while preserving the overall intake structure.

In one or more embodiments, question budget enforcement may prevent interview fatigue by limiting the number of questions per phase and across the entire session. The system may track question counts, patient engagement metrics, and session duration to optimize the balance between clinical completeness and patient experience.

### Alternative Embodiments of Phase Management

In alternative embodiments, the Multi-Phase Intake Controller (1200) may implement parallel phase processing, where multiple clinical domains may be explored simultaneously rather than sequentially. This approach may reduce total interview time by identifying natural conversation flows that address multiple domains within single patient responses.

Some embodiments may implement conditional phase skipping based on patient acuity or visit type. For routine follow-up visits, certain phases such as comprehensive problem checklists may be abbreviated or skipped entirely, while emergency presentations may trigger immediate safety-focused phases. Other embodiments may provide patient-controlled phase navigation, allowing patients to indicate their preferred interview structure while maintaining mandatory safety and clinical requirements. This approach may improve patient satisfaction and engagement while preserving clinical rigor.

### Safety Enforcement Layer: Comprehensive Defense-in-Depth Architecture

In one or more embodiments, a Defense-in-Depth Safety Enforcement Layer may implement a sophisticated architecture with multiple independent safety detection mechanisms. The system architecture may comprise the Primary AI Safety Classifier, the Secondary Deterministic Pattern Detector, the Always-Ask Registry, the Completion Gate Enforcer, and the Safety Corroboration Module.

In one or more embodiments, the Primary AI Safety Classifier may leverage advanced natural language processing to perform semantic analysis of patient responses, distinguishing between figurative expressions and genuine safety concerns. The classifier may assign structured safety assessments including safety levels (LOW, MODERATE, HIGH, CRITICAL), safety categories (suicidal ideation, homicidal thoughts, abuse disclosures, medical emergencies, substance issues), confidence scores, and requires-immediate-action flags.

In one or more embodiments, the Secondary Deterministic Pattern Detector may operate as an independent backup system using hardcoded pattern matching against curated keyword libraries. This detector may scan every patient response for specific phrases indicating safety risks, including passive expressions of suicidal ideation that AI semantic analysis might miss, such as “nothing matters anymore,” “everyone would be better off,” “I just want to sleep forever,” or “there's no point in trying.”

### Technological Advantages of Defense-in-Depth Safety

The defense-in-depth approach may provide critical technological advantages for clinical safety. First, redundant detection mechanisms may significantly reduce false negatives in safety screening by implementing independent verification pathways. If the AI classifier misses a subtle safety concern, the deterministic pattern detector may still flag the issue for clinical attention. In one or more embodiments, the system may implement safety corroboration requirements that prevent AI hallucination of safety coverage. When the AI claims that a safety-critical domain has been addressed, the Safety Corroboration Module may cross-check this claim against actual conversation history and domain state records. If corroborating evidence is not found, the system may force explicit safety questions to be asked.

In one or more embodiments, the Always-Ask Registry (306) may maintain an immutable list of questions that must be posed in specific contexts, regardless of AI recommendations. This registry may be configured by clinical administrators and may not be overridden by AI decisions, patient requests, or system configurations.

### Alternative Safety Enforcement Embodiments

Alternative embodiments may implement graduated safety responses based on detected severity levels. Rather than binary safety flagging, the system may implement tiered interventions: low-level concerns may trigger additional follow-up questions, moderate concerns may require supervisor notification, high concerns may initiate immediate clinical contact, and critical concerns may trigger emergency protocols.

Some embodiments may provide configurable safety sensitivity levels that can be adjusted based on clinical setting, patient population, or regulatory requirements. Pediatric settings may implement more sensitive detection thresholds, while routine primary care may use standard sensitivity levels. Other embodiments may implement machine learning-enhanced pattern detection that continuously updates keyword libraries based on missed safety events or false positive patterns, improving detection accuracy over time while maintaining deterministic backup capabilities.

### Domain Enforcement and Completion Gate Architecture

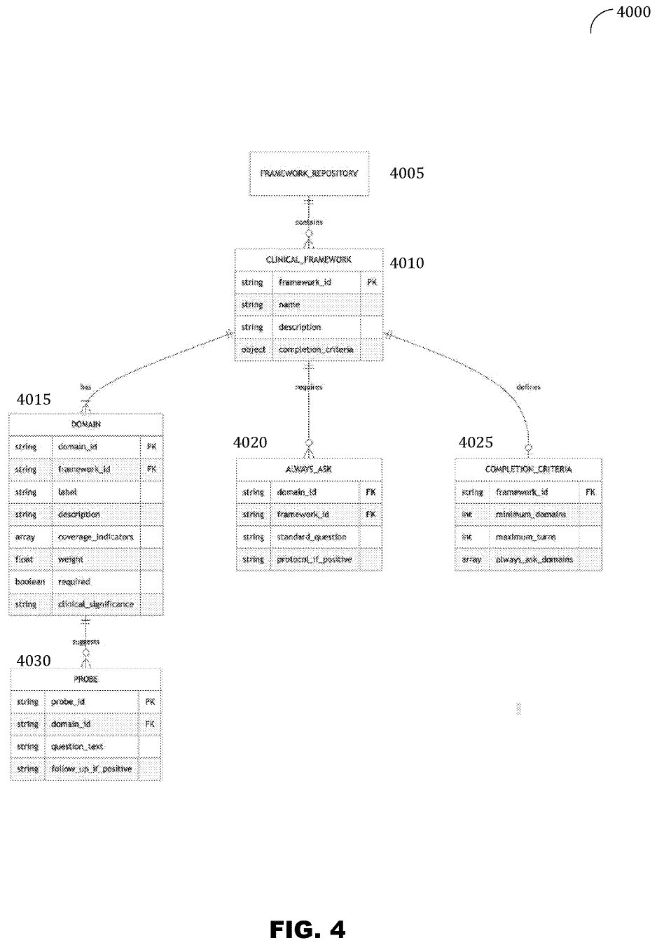

Referring to FIG. 4, the system may implement a sophisticated flowchart of domain enforcement mechanisms with completion gates. The Always-Ask Domain Enforcement may operate through a structured decision tree that evaluates domain coverage status, applies completion gate logic, and enforces mandatory questioning requirements. In one or more embodiments, the completion gate mechanism may implement hard stops that prevent interview termination when safety-critical domains remain unaddressed. When the AI indicates that an interview should be complete, the Completion Gate Enforcer may systematically verify that all always-ask domains have transitioned to ANSWERED or INFERRED states in the Domain Tracking State Machine.

Moreover, FIG. 4 may illustrate a data structure diagram (4000) showing the clinical framework repository entity-relationship schema that defines how clinical frameworks, domains, probes, completion criteria, and always-ask requirements are structured and related within the system. The Framework Repository (4005) may serve as the top-level container for all clinical framework definitions. The Clinical Framework table (4010) may represent the central entity defining each clinical questioning framework, storing the framework identifier, name, description, and completion criteria object. Each clinical framework may define multiple related entities through structured relationships. The Domain table (4015) may store individual clinical domains associated with each framework, including domain identifiers, labels, descriptions, coverage indicators arrays, weighted priority values, required/optional flags, and clinical significance ratings. The weighted coverage indicators may enable the system to prioritize domains based on clinical importance and patient-specific factors. The Probe table (4030) may contain suggested questions for each domain, storing probe identifiers, associated domain identifiers, question text, and follow-up protocols for positive responses. These probes may guide the AI in generating contextually appropriate questions while maintaining clinical framework compliance. The Always-Ask table (4020) may define mandatory safety-critical questions that must be posed in specific contexts, storing domain identifiers, framework identifiers, standard question text, and protocols for positive responses. This table may enforce completion gate requirements that prevent interview termination until all always-ask domains are addressed. The Completion Criteria table (4025) may specify the requirements for framework completion, including framework identifiers, minimum domain thresholds indicating how many domains must be covered, maximum turn limits to prevent interview fatigue, and arrays of always-ask domain identifiers that cannot be bypassed. These completion criteria may enable the Completion Gate Enforcer to verify that all mandatory requirements are satisfied before allowing interview conclusion.

In one or more embodiments, the entity relationships may use crow's-foot notation indicating one-to-many relationships: one clinical framework may have multiple domains, one domain may have multiple probes, one framework may define completion criteria, and one framework may specify multiple always-ask requirements. This normalized schema may support efficient retrieval, modification, and validation of clinical framework definitions across diverse clinical contexts.

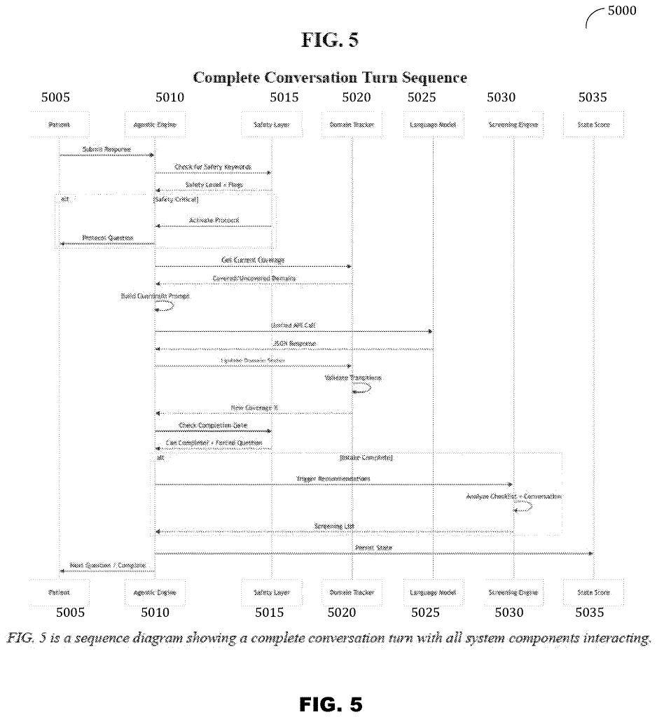

FIG. 5 is a sequence diagram showing a complete conversation turn with all system components interacting. FIG. 5 may illustrate a sequence diagram (5000) showing a complete conversation turn with all system components interacting in a coordinated workflow from patient input submission through response generation, safety validation, domain tracking updates, and state persistence. The Patient component (5005) may submit a response to initiate the conversation turn, sending patient-provided text input to the Agentic Engine for processing. The Patient component may receive the next question or completion notification at the end of the sequence. The Agentic Engine (5010) may serve as the central orchestrator coordinating all interactions between system components. The Agentic Engine may receive the patient response, may check for safety keywords by communicating with the Safety Layer, may retrieve current domain coverage status from the Domain Tracker, may build guardrails prompts incorporating safety constraints and coverage requirements, may execute a unified API call to the Language Model, may receive the JSON response containing the AI-generated question, may update domain states based on the response content, may check completion gate criteria, and may return the next question or completion signal to the Patient. The Safety Layer (5015) may receive safety keyword check requests from the Agentic Engine and may analyze patient input for safety-critical indicators. The Safety Layer may return safety level classifications and flags indicating whether safety protocols must be activated. When safety-critical content is detected, the Safety Layer may activate protocol questions that override normal conversation flow to address immediate clinical concerns. The Domain Tracker (5020) may maintain the current coverage status for all clinical domains throughout the conversation. The Domain Tracker may receive coverage status requests from the Agentic Engine and may return arrays of covered and uncovered domains. The Domain Tracker may validate state transitions to ensure that domain status changes are supported by actual conversation evidence. The Domain Tracker may update domain states after each conversation turn, may compute new coverage percentages, may check completion gate requirements, and may return completion eligibility status along with any forced questions that must be asked before interview conclusion. The Language Model (5025) may receive unified API calls from the Agentic Engine containing the constructed prompt with patient context, conversation history, safety constraints, domain coverage status, and generation instructions. The Language Model may generate contextually appropriate follow-up questions and may return structured JSON responses containing the question text, reasoning, domain coverage indicators, and metadata. The Screening Engine (5030) may be triggered when the intake interview reaches completion. The Screening Engine may receive a trigger recommendations request from the Agentic Engine, may analyze the complete conversation history against clinical screening checklists, and may generate screening lists containing recommended tests, referrals, and clinical actions based on the patient's disclosed symptoms and medical history. The State Store (5035) may persist conversation state throughout the interaction sequence. The State Store may receive persist state commands from the Agentic Engine and may save conversation turns, domain tracking status, safety flags, and session metadata to enable conversation continuity across system restarts and to support retrospective analysis. The complete conversation turn sequence may begin with patient input submission (5005 to 5010), proceed through safety validation (5010 to 5015), domain coverage retrieval (5010 to 5020), prompt construction and AI generation (5010 to 5025), domain state updates and completion checking (5010 to 5020), screening trigger evaluation (5010 to 5030), state persistence (5010 to 5035), and conclude with delivery of the next question or completion notification back to the patient (5010 to 5005).

### Technological Advantages of Completion Gates

Completion gates may provide several critical technological advantages. First, they may prevent premature interview termination due to AI limitations, patient reluctance, or system errors. The gates may operate independently of AI decision-making, ensuring that clinical requirements take precedence over conversational flow. In one or more embodiments, the system may implement domain prioritization logic that ensures high-priority safety domains are addressed before lower-priority clinical domains. This approach may optimize clinical risk management while maintaining interview efficiency. In further embodiments, completion gates may provide audit trails that document exactly which domains were covered, how they were addressed, and what evidence supports their completion status. This documentation may be critical for clinical liability and quality assurance purposes.

### Alternative Completion Gate Embodiments

Alternative embodiments may implement soft completion gates that provide warnings rather than hard stops when domains remain unaddressed. These systems may allow clinical overrides while maintaining documentation of incomplete coverage. Some embodiments may provide time-based completion gate relaxation, where mandatory requirements may be reduced after extended interview sessions to prevent patient fatigue while maintaining core safety requirements. Other embodiments may implement patient-specific completion gate customization based on medical history, risk factors, or clinical setting, allowing more targeted and efficient safety screening.

### Safety Keyword Detection and Severity Escalation

In one or more embodiments, the system may implement a comprehensive decision tree for safety keyword detection and severity escalation. The decision tree may process patient input through multiple evaluation pathways: direct keyword matching, contextual phrase analysis, sentiment evaluation, and severity classification. In one or more embodiments, the keyword detection system may organize safety terms into hierarchical categories with associated severity levels. Explicit suicidal statements may trigger immediate CRITICAL classification, while passive expressions may result in HIGH classification requiring follow-up questioning. The system may implement contextual analysis to distinguish between metaphorical usage (“I'm dying to see that movie”) and genuine safety concerns (“I feel like I'm dying”).

### Technological Advantages of Hierarchical Safety Detection

The hierarchical approach may provide several technological advantages. First, it may enable graduated responses appropriate to detected severity levels, avoiding unnecessary escalation for low-risk expressions while ensuring appropriate intervention for high-risk disclosures. In one or more embodiments, the system may implement contextual disambiguation that reduces false positives by analyzing surrounding conversation context, patient history, and linguistic patterns to distinguish between genuine safety concerns and casual expressions. In further embodiments, the decision tree may provide transparent escalation logic that can be audited, validated, and adjusted by clinical administrators to meet specific organizational requirements and regulatory standards.

### Alternative Safety Detection Embodiments

Alternative embodiments may implement machine learning-enhanced safety detection that continuously improves classification accuracy based on clinical outcomes and provider feedback. These systems may learn from false positives and missed detections to refine keyword libraries and severity thresholds. Some embodiments may provide multi-language safety detection capabilities that can identify safety concerns across different languages and cultural expressions, expanding system accessibility for diverse patient populations. Other embodiments may implement real-time provider notification systems that immediately alert clinical staff when high-severity safety concerns are detected, enabling rapid clinical intervention.

### Technological Advantages of State Machine Architecture

The state machine approach may provide several critical technological advantages. First, it may ensure deterministic domain tracking that cannot be influenced by AI hallucination or inconsistent decision-making. Each domain's status may be explicitly tracked and validated against actual conversation history. In one or more embodiments, the system may implement state transition validation that prevents invalid state changes and maintains data integrity throughout the interview process. This validation may prevent domains from being marked as complete without appropriate evidence. In one or more embodiments, the state machine may provide comprehensive audit capabilities that document exactly when and how each domain was addressed, supporting clinical documentation and quality assurance requirements.

### Alternative State Management Embodiments

Alternative embodiments may implement probabilistic state transitions that assign confidence scores to domain coverage rather than binary completion status. These systems may provide more nuanced coverage assessment while maintaining clear completion criteria. Some embodiments may provide hierarchical domain structures where high-level domains may be subdivided into specific sub-domains, allowing more granular coverage tracking and targeted follow-up questioning. Other embodiments may implement temporal state management that tracks not only whether domains were covered but also when they were addressed and how recently information was updated, supporting longitudinal care coordination.

### System Integration and Operational Advantages

The integrated system architecture may provide numerous operational advantages over traditional clinical intake methods. The combination of multi-phase orchestration, defense-in-depth safety enforcement, and deterministic domain tracking may enable healthcare organizations to conduct comprehensive, safe, and efficient patient intake interviews while maintaining clinical rigor and regulatory compliance. In one or more embodiments, the system may reduce clinical workload by automating routine questioning while ensuring that critical safety and clinical domains are systematically addressed. Providers may receive structured, comprehensive intake documentation that highlights key clinical findings and safety concerns, enabling more focused and effective clinical encounters. In one or more embodiments, the architecture may support scalable deployment across diverse clinical settings, from primary care practices to emergency departments to behavioral health clinics, with configurable frameworks, safety protocols, and documentation requirements tailored to specific clinical contexts and regulatory environments.

These technological innovations may collectively enable a new paradigm of AI-assisted clinical intake that maintains the thoroughness and safety of traditional clinical interviews while providing the efficiency and consistency advantages of automated systems. The embodiments described may be implemented individually or in various combinations to provide flexible, safe, and effective clinical intake capabilities that enhance both patient experience and clinical outcomes.

### Computing Device

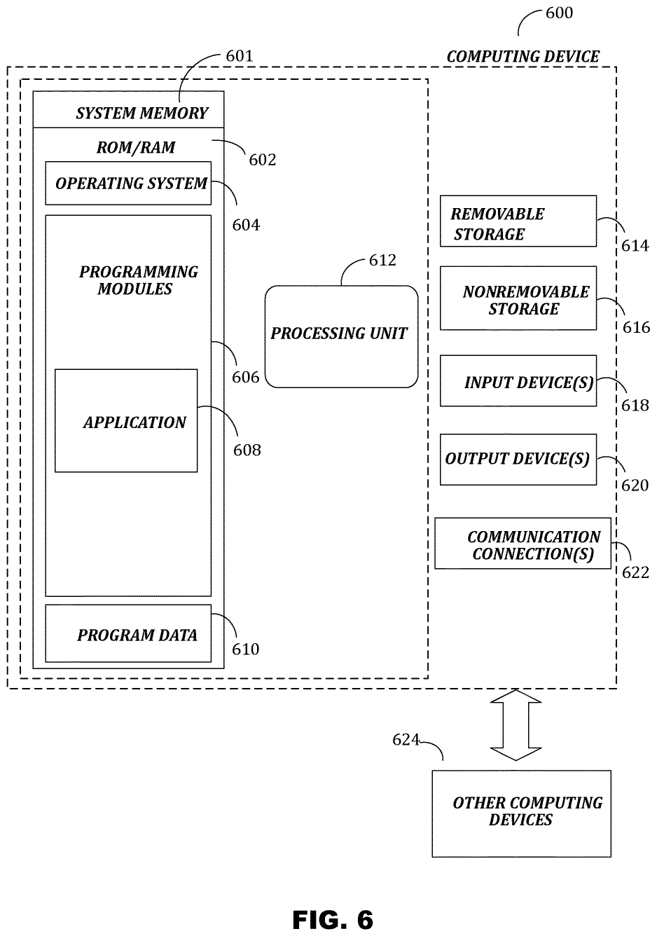

FIG. 6 is a block diagram illustrating an exemplary computing device 600 that may be used to implement various aspects of the invention. The device 600 includes a processing unit 612, a system memory 601, and a system bus that couples various system components including the system memory to the processing unit 612. The system memory 601 includes read-only memory (ROM) 602 and random access memory (RAM) 602. A basic input/output system (BIOS), containing the basic routines that help to transfer information between elements within the computer 600, such as during start-up, is typically stored in ROM 602. The computer 600 may include a removable storage 614 which may be a hard disk drive for reading from and writing to a hard disk, a non-removable storage 616 which may be a magnetic disk drive for reading from or writing to a removable magnetic disk, and an optical disk drive for reading from or writing to a removable optical disk such as a CD-ROM or other optical media. The removable storage 614, non-removable storage 616, and optical disk drive are connected to the system bus as well as various interfaces including but not limited to a hard disk drive interface, a magnetic disk drive interface, and an optical drive interface, respectively.

A number of program modules may be stored on the hard disk, magnetic disk, optical disk, ROM 602 or RAM 602, including an operating system 604, one or more application programs 606, other program modules 608, and program data 610. A user may enter commands and information into the computer 600 through input devices 618 such as a keyboard and pointing device, such as a mouse. The computing device may display or communicate to the user via one or more output devices 620. The computer 600 may connect to various communication terminals, via wired and wireless communication connections 622. The computer 600 may operate in a networked environment using logical connections to one or more remote computers 624, such as a remote computer. The remote computer may be another personal computer, a server, a router, a network PC, a peer device or other common network node, and typically includes many or all of the elements described above relative to the computer 600.

### CONCLUSION

Embodiments may be implemented as methods, systems, or articles of manufacture. The computer program product may be a computer storage media readable by a computer system and encoding a computer program of instructions. Throughout this application, various publications may be referenced. The disclosures of these publications in their entireties are hereby incorporated by reference. Nothing herein is to be construed as an admission that the present disclosure is not entitled to antedate such publication. The patentable scope is defined by the claims. No claim element is construed under 35 U.S.C. § 112(f) unless expressly recited using “means for.” Where a method claim does not specifically state steps are to be limited to a specific order, no order is inferred. While aspects may be described and claimed in a particular statutory class, each aspect may be described and claimed in any statutory class.

Embodiments of the present disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the present disclosure may also be practiced using other technologies capable of performing logical operations such as, for example, AND, OR, and NOT, including but not limited to mechanical, optical, fluidic, and quantum technologies. In addition, embodiments of the present disclosure may be practiced within a general purpose computer or in any other circuits or systems.

Embodiments of the present disclosure may be implemented as a computer process (method), a computing system, or as an article of manufacture, such as a computer program product or computer readable media. The computer program product may be a computer storage media readable by a computer system and encoding a computer program of instructions for executing a computer process. Accordingly, the present disclosure may be embodied in hardware and/or in software (including firmware, resident software, micro-code, etc.).

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list) may include the following: an electrical connection having one or more wires, a portable computer diskette, a random access memory (RAM), a read-only memory (ROM), an erasable programmable read-only memory (EPROM or Flash memory), an optical fiber, and quantum computing elements.

It should be understood that the embodiments described herein are exemplary and that a person skilled in the art may make many variations and modifications without departing from the spirit and scope of the present disclosure. All such variations and modifications are intended to be included within the scope of the present disclosure as defined in the appended claims. While illustrative embodiments have been shown and described, variations and alternative embodiments may occur to those skilled in the art. Such variations and alternative embodiments may be made without departing from the scope of the present disclosure as defined in the claims.

The patentable scope of the present disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Insofar as the description above and the accompanying drawings disclose any additional subject matter that is not within the scope of the claims below, the disclosures are not dedicated to the public and the right to file one or more applications to claim such additional disclosures is reserved.

What is claimed is:

1. A computer-implemented method for conducting clinical patient intake using artificial intelligence, comprising:
receiving, by a processor, a chief complaint from a patient through a user interface;
selecting, by the processor, a clinical assessment framework from a plurality of stored frameworks based on the chief complaint;
generating, by the processor, contextual clinical questions using a language model; and
generating clinical documentation from the intake session.

2. The method of claim 1, further comprising:
initializing a domain tracking state machine with a discrete domain state record for each clinical domain in the selected framework, each domain state record having an initial status of NOT_STARTED; and
tracking each clinical domain through discrete states comprising at least NOT_STARTED, ASKED, ANSWERED, and INFERRED.

3. The method of claim 2, wherein selecting the clinical assessment framework comprises keyword matching with scoring against the chief complaint, and wherein each framework comprises a set of clinical domains with coverage indicators, priority values, and required flags.

4. The method of claim 2, further comprising:
generating guardrails comprising explicit domain coverage status from the domain tracking state machine, the guardrails comprising lists of covered domains with source attribution and uncovered domains with priority ordering; and
injecting the guardrails into a prompt for the language model.

5. The method of claim 4, wherein generating contextual clinical questions comprises generating, in a single API call to the language model, a unified response comprising a next clinical question, a list of covered domain identifiers, a completion indicator, and a safety level.

6. The method of claim 1, further comprising performing safety classification on patient input using a multi-layered approach comprising:
performing primary safety classification using the language model, returning a structured assessment comprising safety level, safety category, confidence, and a requires-immediate-action flag; and
activating, independently of the primary safety classification, a secondary deterministic pattern detector scanning patient input against keyword libraries organized across a plurality of safety categories.

7. The method of claim 6, further comprising enforcing mandatory safety screenings by blocking intake completion through a hard completion gate when any safety-critical domain classified as always-ask remains in a NOT_STARTED or ASKED state in the domain tracking state machine, regardless of the completion indicator from the language model.

8. The method of claim 7, wherein enforcing safety screenings further comprises a corroboration requirement wherein AI-reported coverage of a safety-critical domain may not be accepted unless corroborating evidence keywords are found within a predetermined number of the most recent question-and-answer pairs in the conversation history.

9. The method of claim 1, wherein generating clinical documentation comprises applying clinical language transformation rules to convert conversational patient speech into professional medical terminology.

10. An integrated clinical intake system comprising:
a processor; and
memory storing instructions that, when executed by the processor, cause the processor to implement:
an agentic intake engine configured to dynamically select clinical assessment frameworks from a clinical framework repository based on chief complaint keyword analysis and to generate contextual questions through unified API calls with guardrails injection from a domain tracking state machine;
a defense-in-depth safety enforcement layer comprising a primary AI-powered semantic safety classifier and a secondary deterministic pattern detector scanning patient input against curated keyword libraries organized across a plurality of safety categories, with a completion gate enforcer implementing hard completion gates that prevent intake completion when safety-critical domains remain unaddressed regardless of AI-generated completion signals;
a domain tracking state machine maintaining each clinical domain in a discrete state selected from NOT_STARTED, ASKED, ANSWERED, INFERRED, SKIPPED, and BLOCKED, with a state transition validator enforcing valid state transitions;
an automated clinical document generation system configured to produce at least one of a SOAP note and a behavioral health intake report with clinical language transformation converting colloquial patient speech into professional medical terminology; and
a context-aware empathy injection system configured to scan AI-generated output for empathy markers, to inject context-appropriate empathetic prefixes when empathy markers are absent, and to strip empathetic prefixes before comparing question signatures for redundancy detection.

11. The system of claim 10, further comprising an EHR context injection system configured to authenticate with external electronic health record systems via FHIR R4 interfaces, retrieve patient medications, allergies, conditions, and prior encounters, persist the retrieved data as a session state snapshot surviving serialization, and inject the data into AI prompts to enable clinically informed questioning.

12. The system of claim 10, wherein updates generated for insertion into an electronic health record are stored in a staged non-finalized state separate from the permanent medical record, and wherein the system requires explicit review and approval by a licensed healthcare provider before committing the updates to the permanent medical record, the staging subsystem maintaining version control, timestamps, provider identifiers, and audit logs.

13. The system of claim 10, further comprising an adaptive readability and linguistic personalization subsystem configured to compute standardized readability metrics for patient-generated text input and AI-generated questions, to maintain a user linguistic preference profile per patient, to constrain AI-generated output to a target readability range associated with the patient's profile, and to operate independently from the defense-in-depth safety enforcement layer and the domain tracking state machine such that personalization adjustments may not suppress required safety questions or domain coverage enforcement.

14. The system of claim 10, further comprising presenting the patient with a selection of virtual agent personalities representing distinct communication styles, each personality layered on mandatory base guardrails enforcing safety protocols, clinical framework compliance, and empathy requirements.

15. The system of claim 10, further comprising a medication reconciliation system configured to extract structured medication data from patient speech using pattern matching for dosage values and frequency terms, reconcile extracted medications against stored patient records, classify each medication as confirmed, new, discontinued, changed, or needs_followup, and generate targeted follow-up probe questions for medications with missing required details.

16. A non-transitory computer-readable medium storing instructions that, when executed by a processor, cause the processor to:
receive patient input through a conversational user interface;
dynamically select a clinical assessment framework from a plurality of stored frameworks based on patient chief complaint keyword matching with scoring;
maintain a domain tracking state machine with discrete domain states comprising NOT_STARTED, ASKED, ANSWERED, INFERRED, SKIPPED, and BLOCKED, and with enforced state transitions;
inject explicit domain coverage status from the domain tracking state machine into AI prompts as guardrails;
enforce mandatory safety screenings through hard completion gates that may not be bypassed by AI-generated completion signals, patient requests, or configuration changes;
apply defense-in-depth safety detection using both primary AI semantic classification and secondary deterministic pattern matching against keyword libraries organized across a plurality of safety categories;
generate clinical documentation from the patient intake session with clinical language transformation; and
present a selection of virtual agent personalities to the patient, each personality layered on mandatory base guardrails enforcing safety protocols, clinical framework compliance, and empathy requirements.

17. The computer-readable medium of claim 16, wherein the instructions further cause the processor to manage intake through a plurality of defined phases comprising at least patient type determination, structured intake, substance history, problem checklist, medication reconciliation, freeform AI interview, closeout, and completion, and bridge context between phases by serializing data from prior phases into AI prompts.

18. The computer-readable medium of claim 16, wherein the instructions further cause the processor to receive problem checklist data with symptom severity ratings on a discrete scale, map symptom categories to clinical screenings using category-to-screening mapping tables with category-specific severity thresholds, perform parallel problem checklist analysis and conversation keyword scanning, apply demographic adjustments, and generate prioritized screening recommendations with documented evidence comprising triggering problems and severity ratings.

19. The computer-readable medium of claim 16, wherein the instructions further cause the processor to compute a readability score for AI-generated output using a standardized readability metric, maintain a user linguistic preference profile comprising at least an average patient readability score, a preferred output readability range, historical satisfaction metrics, and lexical complexity preference, and when the readability score falls outside a predefined tolerance band relative to the patient's preferred output readability range, perform at least one of: requesting regeneration from the language model with an explicit readability constraint, or applying a post-processing rewrite to adjust lexical complexity, sentence length, and syntactic structure.

20. The computer-readable medium of claim 16, wherein the instructions further cause the processor to detect a proxy scenario by analyzing patient input against categorized pattern libraries comprising pediatric, geriatric, spouse, caregiver, and guardian categories, extract a proxy context comprising a patient reference, and adjust all subsequent question phrasing to use third-person patient references corresponding to the detected relationship.
